(19) United States
(12) Patent Application Publication
(10) Pub. No.: US 2022/0213086 A1
(43) Pub. Date: Jul. 7, 2022

(54) AZOLE COMPOUNDS AS IRAK INHIBITORS, PREPARATION METHODS AND MEDICINAL USES THEREOF

(71) Applicant: Eternity Bioscience Inc., Cranbury, NJ (US)

(72) Inventors: Hongbin Li, Newtown, CT (US); Jian Liu, Edison, NJ (US); Bin Cao, Cranbury, NJ (US); Linghang Zhuang, Chalfont, PA (US); Tongyuan Li, Fort Lee, NJ (US); Suxing Liu, Edison, NJ (US)

(21) Appl. No.: 17/561,894

(22) Filed: Dec. 24, 2021

Related U.S. Application Data
(60) Provisional application No. 63/130,628, filed on Dec. 25, 2020.

(51) Int. Cl. C07D 417/12

(52) U.S. Cl. CPC C07D 417/12

(57) ABSTRACT

This application discloses azole compounds of formula (I) useful as IRAK inhibitors and therapeutic agents for treatment of IRAK, especially IRAK-4, mediated disease or disorders, including autoimmune diseases, cancers, neurodegenerative disorders, viral diseases, and inflammatory disorders, hereditary disorders, and so on. The application also discloses pharmaceutical compositions containing these compounds, as well as synthetic methods and medical uses of the compounds.

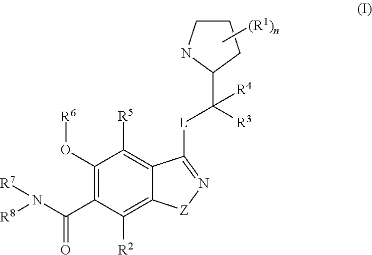

### CROSS-REFERENCE TO RELATED APPLICATION

This application claims priority under 35 U.S.C. § 119(e) to United States Provisional Patent Application No. 63/130,628, filed on Dec. 25, 2020, the disclosure of which is incorporated herein by reference in its entirety.

### FIELD OF THE DISCLOSURE

The present disclosure relates to compounds useful in the inhibition of the function of interleukin-1 receptor-associated kinase (IRAK) and use of them as therapeutic agents for treatment of diseases and conditions associated with IRAK activities, such as cancers, neurodegenerative disorders; autoimmune diseases, inflammatory diseases, infectious diseases, cardiovascular disorders, CNS disorders, and so on.

### BACKGROUND OF THE DISCLOSURE

As a central signaling mediator, Interleukin-1 receptor-associated kinase 4 (IRAK4) plays crucial roles in transducing the responses of the interleukin-1 (IL-1) receptors and Toll-like receptors (TLR). IRAK4 overactivation is linked with different types of human autoimmune and inflammatory diseases. A number of studies reported that all TLR/IL-1Rs with known function are involved in host defense mechanisms, either by the recognition of pathogens or as receptors for inflammatory cytokines. They play a vital role in both innate and adaptive immunity in mammals, and the signaling cascades mediated by these receptors are associated with various human diseases. IRAK proteins are key components in the signal transduction pathways mediated by interleukin-1 receptor (IL-IR), interleukin-18 receptor (IL-18R), and Toll-like receptors (TLRs).

IRAK family consists of four members, including TEAK-1, IRAK-2, IRAK-M, also denoted IRAK-3, and IRAK-4. Among them, IRAK1 and IRAK4 proteins are catalytically active kinases, whereas IRAK2 and IRAK3 are inactive pseudokinases (Jain et al., Front. Immunol., 2014, 5(553): 1-8). IRAK-4 belongs to the serine/threonine kinase and is the best characterized member of the IRAK family. IRAK-4 is involved in signaling innate immune responses from both TLRs and IL-1 receptors. Innate immunity detects pathogens through the recognition of pathogen-associated molecular patterns (PAMPs) by TLRs. TLRs recognize conserved structures of both microbes and endogenous molecules. The cell surface TLRs recognize bacterial and fungal components, whereas TLRs recognizing viral or microbial nucleic acids are localized into intracellular membranes such as endosomes and phagosomes. Upon ligand binding, these receptors recruit the scaffolding adaptor protein myeloid differentiation primary response gene (88) (MyD88) through the Toll/interleukin-1 (IL-1) receptor (TIR) domain. MyD88 is the universal adaptor used by all TLRs except for TLR3. MyD88 couples to pathways that lead to the activation of transcription factors such as NF-κB (nuclear factor-κB), IRF1 (IFN-regulatory factor 1), IRF5 and IRF7 (Luke A. J., et al., Nat. Rev. Immunol., 2007, 7: 353-364). MyD88 utilizes the death domain to recruit IRAK4, thereby activating its kinase function to phosphorylate IRAK1 and IRAK2 within the myddosome complex. Phosphorylation at multiple sites allows IRAK to dissociate from the myddosome complex and activate the downstream proteins such as TNF receptor-associated factor 6 (TRAF-6), which finally can initiate downstream activation of NF-kB and MAPK signaling pathways, leading to the induction of pro-inflammatory cytokines and chemokines, such as TNF-α IL-6, and IL-8.

Mice homozygous for disruptions in this gene display an essentially normal phenotype, and in overexpression studies, IRAK-2 and IRAK-3 can compensate for IRAK-1 loss in a mutant 293 cell line, furthermore, in vitro assay demonstrated that kinase activity of IRAK1 is not required for IL-1 downstream signaling (Knop and Martin, FEBS Lett, 1999, 448: 81-85; Thomas, J A., et al., J. Immunool., 1999, 163:978-984; Wesche, H. et al., Biol. Chem., 1999, 274:19403-19410; Aravind L., et al., Science, 2001, 291:1279-1284), Both IRAK-4 and MyD88 deficient humans show normal resistance to common fungi, parasites, viruses, and many bacteria, except the susceptibility to bacterial infections, particularly recurrent pyogenic bacterial infections such as Streptococcus infections (Picard, C., et al., Science, 2003, 299:2076-2079; Ku, C-L., et al., J. Exp. Med., 2007, 204(10):2407-2022). Human germline gain-of-function MyD88 mutation which causes the constitutive activation of IRAK4 signaling led to severe arthritis (Sikora et al., J. Allergy Clin. Immunol., 2018, 141(5):1943-1947; Picard, C., et al., Clin. Microbiol. Rev., 2011, 24(3): 490-497; Koziczak-Holbro et al., Arthritis Rheum., 2009, 60(6): 1661-1671), IRAK1-deficient human peripheral blood mononuclear cells (PBMCs) have normal responses to both TLR agonists and IL-113, but the responses in TRAK4 deficient human PBMCs are totally abolished (Mina et al., PNAS, 2017, E514-E523). Mouse knock-out experiments have demonstrated an essential role for IRAK-4 in IL-IR, IL-18R and most TLR signaling, IRAK4-deficient mice also exhibit defective innate immunity and are more susceptible to bacterial infection. IRAK4 kinase dead mice are protected against antigen-induced arthritis. (Suzuki, N., et al., Nature, 2002, 416:750-756; Koziczak-Holbro et al., Arthritis Rheum., 2009, 60(6): 1661-1671).

IRAK4 also plays a central role in tumor growth and progression, Inhibition of IRAK4 is universally toxic towards activated B-cell diffuse large B-cell lymphoma (ABC DLBCL) but not germinal center B-cell (GCB) DLBCL cell lines, mechanistic studies demonstrated that IRAK4 inhibition potently abrogates IRAK4-mediated phosphorylation of IRAK1 and the downstream activation of NT-KB and MAPK signaling pathways resulting from the MyD88 (L265P) mutation in ABC DLBCL, leading to the suppression of secretion of the pro-inflammatory cytokines TNFα, IL-6 and IL-10 by ABC DLBCL cells. Furthermore, aberrant B cell receptor (BCR) signaling in ABC DLBCL is also engaged in tumor growth and progression, and this chronic active BCR signaling can be blocked by inhibiting Bruton's tyrosine kinase (RTK) pharmacologically. Notably, the IRAK4 inhibitors strongly synergized with BTK inhibition in killing multiple ABC DLBCL cell lines. (Ngo et al., Nature, 2011, 470(7332): 115-119; Lim et al., Blood, 2012, 120(21):625).

Toll/IL-1 receptor family members like IRAK4 are central components of host defense mechanisms in a variety of species and IRAK4-mediated signaling, downstream of TLRs and the IL-1 receptor family, bridges both innate and adaptive immunity. Both TLRs and IL-1 pathways have been linked to immune and inflammatory diseases such as chronic inflammatory diseases, including chronic arthritis, atherosclerosis, multiple sclerosis, cancers, and autoimmune disorders including rheumatoid arthritis, lupus, asthma, psoriasis, and inflammatory bowel diseases. Overall, IRAK4 plays a key role in immune and inflammatory responses and is considered as an important potential therapeutic target for autoimmune diseases, inflammatory diseases, and cancer.

Patent applications for compounds that have been published as IRAK4 inhibitors include WO2015150995A1, WO2019089422A1, WO2020113233A1, WO2019133531A1, WO2015048281 A1, WO2017025849A1, WO2017033093 A1, WO2018209012 A1 and WO2019149522A1, However, there is still a need to develop new IRAK, especially IRAK4, inhibitors as therapeutic agents to meet the unmet medical needs.

### SUMMARY OF THE DISCLOSURE

The compounds of this disclosure inhibit the function of IRAK and accordingly may serve as therapeutic agents for the treatment of diseases or disorders, including cancers, neurodegenerative disorders, viral diseases, autoimmune diseases, inflammatory disorders, hereditary disorders, hormone-related diseases, metabolic disorders, conditions associated with organ transplantation, immunodeficiency disorders, destructive bone disorders, proliferative disorders, infectious diseases, conditions associated with cell death, thrombin-induced platelet aggregation, liver diseases, pathologic immune conditions involving T cell activation, cardiovascular disorders, and central nervous system (CNS) disorders.

The present disclosure, in one aspect, provides a compound of formula (I) or a pharmaceutically acceptable salt thereof:

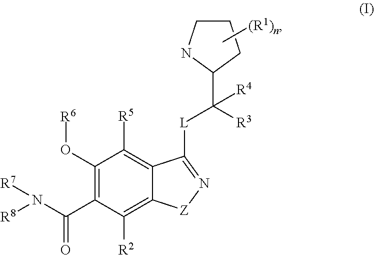

wherein:

Z is O or S;

L is O, S(O)₁, CR^{c}R^{d} or NRⁿ;

R¹ at each occurrence is independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, sometimes more preferably one to three, groups independently, selected from halogen, alkyl, haloalkyl, alkoxy, alkylthio, haloalkoxy, haloalkylthio, hydroxy, hydroxyalkyl, cyano, amino, nitro, —(CH₂)ₛ—NR^{e}R^{f} and cycloalkyl;

or two R¹ together with the attached atoms form a cycloalkyl or heterocyclyl; wherein the cycloalkyl and heterocyclyl are optionally substituted with one or more, preferably one to five, sometimes more preferably one to three, groups independently selected from halogen, alkyl, haloalkyl, alkoxy, alkylthio, haloalkoxy, haloalkylthio, hydroxy, hydroxyalkyl, cyano, amino, nitro and —(CH₂)ₛ—NR^{e}R^{f};

R² and R⁵ are identical or different and are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more, preferably one to five, sometimes more preferably one to three, groups independently selected from halogen, alkyl, haloalkyl, alkoxy, haloalkoxy, hydroxy, hydroxyalkyl, cyano, amino, nitro, —(CH₂)ₛ—NR^{e}R^{f}, cycloalkyl, heterocyclyl, aryl and heteroaryl;

R³, R⁴, R^{c} and R^{d} are identical or different and are independently selected from hydrogen, halogen, alkyl, haloalkyl, alkoxy, alkylthio, haloalkoxy, haloalkylthio, hydroxy, hydroxyalkyl, cyano, amino and —(CH₂)ᵣ—NR^{a}R^{b};

R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, sometimes more preferably one to three, groups independently selected from halogen, alkyl, haloalkyl, alkoxy, alkylthio, haloalkoxy, hydroxy, hydroxyalkyl, cyano, amino, nitro, —(CH₂)ₛ—NR^{e}R^{f} and cycloalkyl;

R⁷, R⁸, R^{a}, R^{b}, R^{e} and R^{f} are identical or different and at each occurrence are independently selected from hydrogen, alkyl, haloalkyl, hydroxyalkyl, —C(═O)OR^{q}, cycloalkyl, heterocyclyl, aryl and heteroaryl;

or R⁷ and R⁸, R^{a} and R^{b}, R^{e} and R^{f} together with the nitrogen to which they are attached form a heterocyclyl; wherein the heterocyclyl is optionally substituted with one or more, preferably one to five, sometimes more preferably one to three, groups independently selected from halogen, alkyl, haloalkyl, alkoxy, haloalkoxy, hydroxy, hydroxyalkyl, oxo, cyano, amino, nitro, cycloalkyl, heterocyclyl, aryl and heteroaryl;

R^{a} is selected from hydrogen, alkyl, haloalkyl, hydroxyalkyl, cycloalkyl, heterocyclyl, aryl and heteroaryl;

R^{q} is selected from hydrogen, alkyl and haloalkyl;

n is 0, 1, 2, 3 or 4;

r is 0, 1, 2 or 3;

s is 0, 1, 2 or 3; and

j is 0, 1 or 2.

In another aspect, this disclosure provides a preparation process of a compound of formula (I) or a pharmaceutically acceptable salt thereof, the preparation process comprising a step of:

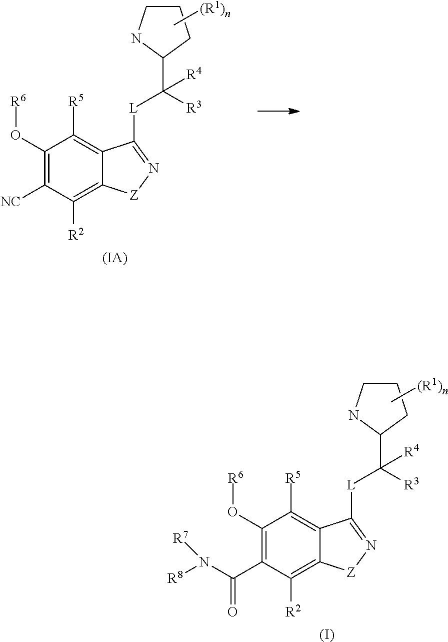

subjecting of a compound of formula (IA) to a hydration reaction to obtain the compound of formula (I), wherein:

R⁷ is hydrogen;

R⁸ is hydrogen; and

Z, L, R¹ to R⁶ and n are each as defined in formula (I).

In another aspect, the present disclosure provides a pharmaceutical composition comprising a compound of formula (I), or a pharmaceutically acceptable salt thereof, and a pharmaceutically acceptable carrier.

In another aspect, the present disclosure provides a method of preventing and/or treating a cancer, a neurodegenerative disorder, a viral disease, an autoimmune disease, an inflammatory disorder, a hereditary disorder, a hormone-related disease, a metabolic disorder; conditions associated with organ transplantation, immunodeficiency disorders, a destructive bone disorder, a proliferative disorder, an infectious disease, a condition associated with cell death, thrombin-induced platelet aggregation, liver disease; pathologic immune conditions involving T cell activation, a cardiovascular disorder, and a CNS disorder, comprising a step of administering to a subject in need thereof a therapeutically effective amount of a compound of formula (I), or a pharmaceutically acceptable salt, or a pharmaceutical composition containing the compound.

In another aspect, the present disclosure also relates to use of a compound of formula (I), or a pharmaceutically acceptable salt thereof, or a pharmaceutical composition containing the compound, in the manufacture of a medicament for treatment of a cancer, a neurodegenerative disorder; a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving cell activation, a cardiovascular disorder; and a CNS disorder.

Other aspects or advantages of the disclosure will be better appreciated in view of the following detailed description, examples, and claims.

### DETAILED DESCRIPTION OF THE DISCLOSURE

In one aspect, the present disclosure provides a compound of formula (I) or a pharmaceutically acceptable salt thereof:

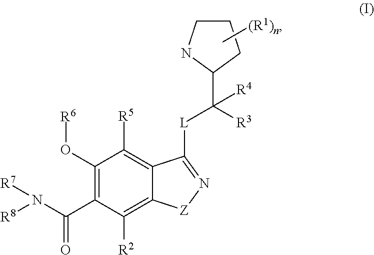

wherein:

Z is O or S;

L is O, S(O)ⱼ, CR^{c}R^{d} or NRⁿ;

R¹ at each occurrence is independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, sometimes more preferably one to three, groups independently selected from halogen, alkyl, haloalkyl, alkoxy, alkylthio, haloalkoxy, haloalkylthio, hydroxy, hydroxyalkyl, cyano, amino, nitro, —(CH₂)ₛ—NR^{e}R^{f} and cycloalkyl;

or two R¹ together with the attached atoms form a cycloalkyl or heterocyclyl; wherein the cycloalkyl and heterocyclyl are optionally substituted with one or more, preferably one to five, sometimes more preferably one to three; groups independently selected from halogen, alkyl, haloalkyl, alkoxy, alkylthio, haloalkoxy, haloalkylthio, hydroxy, hydroxyalkyl, cyano, amino, nitro and —(CH₂)ₛ—NR^{e}R^{f};

R² and R⁵ are identical or different and are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more, preferably one to five, sometimes more preferably one to three, groups independently selected from halogen, alkyl, haloalkyl, alkoxy, haloalkoxy, hydroxy, hydroxyalkyl, cyano, amino, nitro, —(CH₂)ₛ—NR^{e}R^{f}, cycloalkyl, heterocyclyl, aryl and heteroaryl;

R³, R⁴, R^{c} and R^{d} are identical or different and are independently selected from hydrogen, halogen, alkyl, haloalkyl, alkoxy, alkylthio, haloalkoxy, haloalkylthio, hydroxy, hydroxyalkyl, cyano, amino and —(CH₂)ᵣ—NR^{a}R^{b};

R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more, preferably one to five, sometimes more preferably one to three; groups independently selected from halogen, alkyl, haloalkyl, alkoxy, alkylthio, haloalkoxy, hydroxy, hydroxyalkyl, cyano, amino, nitro; —(CH₂)ₛ—NR^{e}R^{f} and cycloalkyl;

R⁷, R⁸, R^{a}, R^{b}, R^{e} and R^{f} are identical or different and at each occurrence are independently selected from hydrogen, alkyl, haloalkyl, hydroxyalkyl, —C(═O)OR^{q}, cycloalkyl, heterocyclyl, aryl and heteroaryl;

or R⁷ and R⁸, R^{a} and R^{b}, R^{e} and R^{f} together with the nitrogen to which they are attached form a heterocyclyl; wherein the heterocyclyl is optionally substituted with one or more, preferably one to five, sometimes more preferably one to three, groups independently selected from halogen, alkyl, haloalkyl, alkoxy, haloalkoxy, hydroxy, hydroxyalkyl, oxo, cyano, amino, nitro, cycloalkyl, heterocyclyl; aryl and heteroaryl;

Rⁿ is selected from hydrogen, alkyl, haloalkyl, hydroxyalkyl, cycloalkyl, heterocycyl, aryl and heteroaryl;

R^{q} is selected from hydrogen, alkyl and haloalkyl;

n is 0, 1, 2, 3 or 4;

r is 0, 1, 2 or 3;

s is 0, 1, 2 or 3; and

j is 0, 1 or 2.

In some embodiments of the disclosure, in the compound of formula (I), R¹ is selected from hydrogen, oxo, R^{1a}, R^{1b}, R^{1c} and R^{1d}; and R⁷ and R⁸ are each hydrogen. Thus, the compound of formula (I), or a pharmaceutically acceptable salt thereof, is a compound of formula (II), or a pharmaceutically acceptable salt thereof:

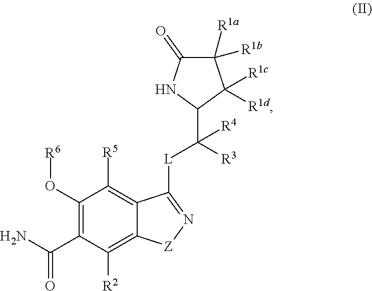

wherein

R^{1a}, R^{1b}, R^{1c} and R^{1d} are identical or different and at each occurrence are independently selected from hydrogen; halogen, alkyl; haloalkyl, alkoxy, alkylthio, haloalkoxy, haloalkylthio, hydroxy; hydroxyalkyl, cyano, amino, —(CH₂)ᵣ—NR^{a}R^{b} and cycloalkyl; and

Z, L, R²-R⁶, r, R^{a} and R^{b} are each as defined in formula (I).

In some embodiments of the disclosure, in the compound of formula (I) or (II), or a pharmaceutically acceptable salt thereof, L is O.

In some embodiments of the disclosure, in the compound of formula (I), L is O; R¹ is selected from hydrogen, oxo; R^{1b} and R^{1d}; and R² to R⁵, R⁷, and R⁸ are each hydrogen; thus, the compound of formula (I), or a pharmaceutically acceptable salt thereof, is a compound of formula (III) or (III′), or a pharmaceutically acceptable salt thereof:

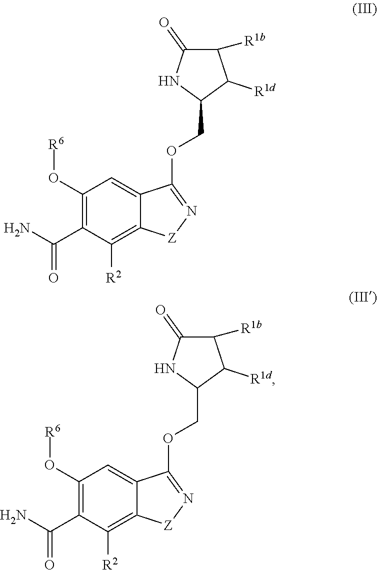

wherein:

R^{1b} and R^{1d} are identical or different and at each occurrence are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b} and cycloalkyl; and

Z, R⁶, R^{a}, R^{b} and r are each as defined in formula (I) above.

In some embodiments of the disclosure, in the compound of formula (I), (II), (III) or (III′), or a pharmaceutically acceptable salt thereof, Z is S.

In some embodiments of the disclosure, in the compound of formula (I) or a pharmaceutically acceptable salt thereof, R¹ at each occurrence is independently selected from hydrogen, halogen, alkyl, haloalkyl, alkoxy, haloalkoxy, hydroxy, hydroxyalkyl, cyano, amino and oxo; sometimes preferably R¹ at each occurrence is selected from hydrogen, halogen. C₁₋₆ alkyl and oxo.

In some embodiments of the disclosure, in the compound of formula (I) or (II), or a pharmaceutically acceptable salt thereof, R² is selected from hydrogen, halogen, alkyl, haloalkyl, alkoxy, haloalkoxy, hydroxy, hydroxyalkyl, cyano and amino; sometimes preferably R² is hydrogen, halogen or C₁₋₆ alkyl.

In some embodiments of the disclosure, in the compound of formula (I) or (II), or a pharmaceutically acceptable salt thereof, R is selected from hydrogen, halogen, alkyl, haloalkyl; sometimes preferably R is hydrogen or C₁₋₆ alkyl.

In some embodiments of the disclosure, in the compound of formula (I) or (II), or a pharmaceutically acceptable salt thereof, R⁴ is selected from hydrogen, halogen, alkyl, haloalkyl; sometimes preferably R⁴ is hydrogen or C₁₋₆ alkyl.

In some embodiments of the disclosure, in the compound of formula (I) or (II), or a pharmaceutically acceptable salt thereof, R⁵ is selected from hydrogen, halogen, alkyl, haloalkyl, alkoxy, haloalkoxy, hydroxy, hydroxyalkyl, cyano and amino; sometimes preferably R⁵ is hydrogen, halogen or C₁₋₆ alkyl.

In some embodiments of the disclosure, in the compound of formula (I), (III) or (AP), or a pharmaceutically acceptable salt thereof, R⁶ is C₁₋₆ alkyl; sometimes preferably R⁶ is methyl.

In some embodiments of the disclosure, in the compound of formula (I) or a pharmaceutically acceptable salt thereof, R⁷ is hydrogen or C₁₋₆ alkyl; sometimes preferably R⁷ is hydrogen.

In some embodiments of the disclosure, in the compound of formula (I) or a pharmaceutically acceptable salt thereof, R⁸ is hydrogen or C₁₋₆ alkyl; sometimes preferably R⁸ is hydrogen.

In some embodiments of the disclosure, in the compound of formula (II), (II) or (III′), or a pharmaceutically acceptable salt thereof, R^{1a} and R^{1b} are identical or different and at each occurrence are independently selected from hydrogen, halogen, alkyl, haloalkyl, alkoxy, haloalkoxy, hydroxy, hydroxyalkyl, cyano, amino and —NR^{a}R^{b}; wherein R^{a} and R^{b} are alkyl; sometimes preferably R^{1a} and R^{1b} are independently selected from hydrogen, halogen and C₁₋₆ alkyl; and sometimes more preferably R^{1a} and R^{1b} are independently hydrogen or fluoro.

In some embodiments of the disclosure, in the compound of formula (II), (III) or (III′), or a pharmaceutically acceptable salt thereof, R^{1c} and R^{1d} are identical or different and at each occurrence are independently selected from hydrogen, halogen, alkyl, haloalkyl, alkoxy, haloalkoxy, hydroxy, hydroxyalkyl, cyano, amino and —NR^{a}R^{b}, wherein R^{a} and R^{b} are alkyl; sometimes preferably R^{1c} and R^{1d} are independently selected from hydrogen, halogen and C₁₋₆ alkyl; and sometimes more preferably R^{1c} and R^{1d} are hydrogen or ethyl.

In some embodiments of the disclosure, in the compound of formula (I) or a pharmaceutically acceptable salt thereof, n is 1 or 3; and sometimes preferably n is 3.

In some embodiments of the disclosure, in the compound of formula (I) or (II), or a pharmaceutically acceptable salt thereof, r is 0, 1 or 2; and sometimes preferably r is 0 or 1.

In some embodiments of the disclosure, in the compound of formula (I), (II), (III) or (III′), or a pharmaceutically acceptable salt thereof, s is 0, 1 or 2; and sometimes preferably s is 0 or 1.

In some embodiments of the disclosure, in the compound of formula (I) or (II), or a pharmaceutically acceptable salt thereof, j is 0 or 1; and sometimes preferably j is 0.

In some embodiments of the disclosure, in the compound of formula (I) or (H), or a pharmaceutically acceptable salt thereof, R^{c} and R^{d} are identical or different and are independently selected from hydrogen, halogen, alkyl and haloalkyl; sometimes preferably R^{c} and R^{d} are identical or different and are independently selected from hydrogen and C₁₋₆ alkyl.

In some embodiments of the disclosure, in the compound of formula (I) or (II), or a pharmaceutically acceptable salt thereof, Rⁿ is selected from hydrogen and C₁₋₆ alkyl; sometimes preferably Rⁿ is hydrogen.

As a person of ordinary skill in the art would understand, any and all plausible combinations of the embodiments disclosed herein, in particular, with regard to the definitions of any substituents, e.g., Z, L, R¹-R⁸, n, R^{1a}, R^{1b}, R^{1c} and R^{1d}, etc. in the compounds of formula (I), (II) and (III) are all encompassed by the present disclosure.

For example, in one embodiment, the present disclosure provides a compound of formula (I), or a pharmaceutically acceptable salt, solvate, or prodrug thereof, wherein:

L is O;

R¹ at each occurrence is independently selected from hydrogen, halogen, alkyl, haloalkyl, alkoxy, haloalkoxy, hydroxy, hydroxyalkyl, cyano, amino and oxo;

R² and R⁵ are each independently selected from hydrogen, halogen, alkyl, haloalkyl, alkoxy, haloalkoxy, hydroxy, hydroxyalkyl, cyano and amino;

R³ and R⁴ are each independently selected from hydrogen, halogen, alkyl, haloalkyl;

R⁶ is C₁₋₆ alkyl; and

R⁷ and R⁸ are independently hydrogen or C₁₋₆ alkyl.

In another embodiment, sometimes preferred, the present disclosure provides a compound of formula (I), or a pharmaceutically acceptable salt, solvate, or prodrug thereof, wherein:

L is O;

Z is S;

R¹ at each occurrence is selected from hydrogen, halogen, C₁₋₆ alkyl and oxo;

R² and R⁵ are independently hydrogen, halogen or C₁₋₆ alkyl;

R³ and R⁴ are independently hydrogen or C₁₋₆ alkyl;

R⁶ is methyl; and

R⁷ and R⁸ are each hydrogen.

Exemplified compounds of the disclosure include, but are not limited to:

| Example | |
|---|---|
| No. | Compound structure and name |
| 1 | 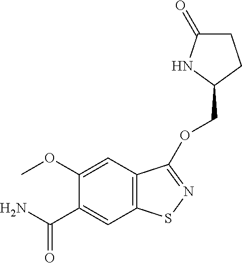 [SMILES: COc1cc2c(OC[C@@H]3CCC(=O)N3)nsc2cc1C(N)=O] |
| | 1 |
| | (S)-5-methoxy-3-((5-oxopyrrolidin-2-y1)methoxy) |
| | benzo[d]isothiazole-6-carboxamide 1 |
| | 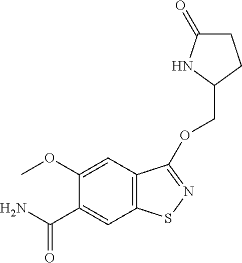 [SMILES: COc1cc2c(OCC3CCC(=O)N3)nsc2cc1C(N)=O] |
| 2 | 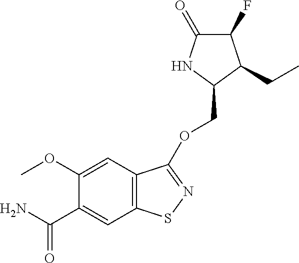 [SMILES: CC[C@@H]1[C@H](F)C(=O)N[C@@H]1COc1nsc2cc(C(N)=O)c(OC)cc12] |
| | 2 |
| | (3-(((2S,3S,4S)-3-ethyl-4-fluoro-5-oxopyrrolidin- |
| | 2-yl)methoxy)-5-methoxybenzo[d]isothiazole-6- |
| | carboxamide 2 |
| | 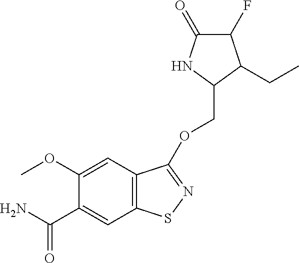 [SMILES: CCC1C(COc2nsc3cc(C(N)=O)c(OC)cc23)NC(=O)C1F] |

In one aspect, the present disclosure provides a compound of formula (IA) or pharmaceutically acceptable salt thereof:

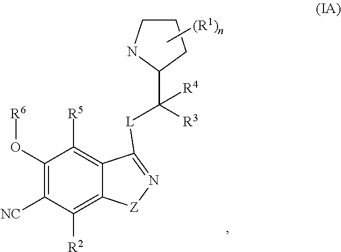

wherein Z, L, R¹-R⁶ and n are each as defined in formula (I).

In one aspect, the present disclosure provides a compound of formula (IIA) or pharmaceutically acceptable salt thereof:

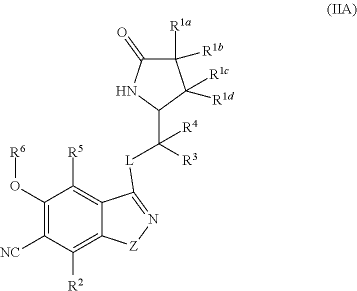

wherein Z, L, R²-R⁶ and R^{1a}-R^{1d} are each as defined in formula (II).

In one aspect, the present disclosure provides a compound of formula (IIIA) or) (III′A), or pharmaceutically acceptable salt thereof:

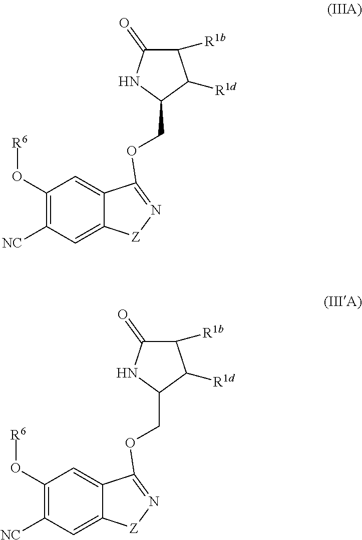

wherein Z, R^{1b}, R^{1d} and R⁶ are each as defined in formula (III).

Exemplified compounds of the disclosure include, but are not limited to:

| Example | |
|---|---|
| No. | Compound structure and name |
| 1h | 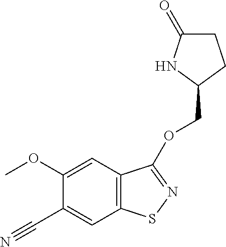 [SMILES: COc1cc2c(OC[C@@H]3CCC(=O)N3)nsc2cc1C#N] |
| | 1h |
| | (S)-5-methoxy-3-((5-oxopyrrolidin-2-yl) |
| | methoxy)benzo[d]isothiazole-6-carbonitrile 1h |
| | 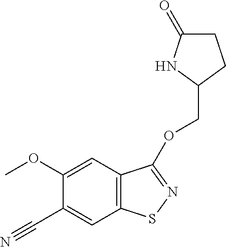 [SMILES: COc1cc2c(OCC3CCC(=O)N3)nsc2cc1C#N] |
| 2c | 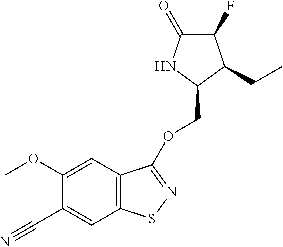 [SMILES: CC[C@@H]1[C@H](F)C(=O)N[C@@H]1COc1nsc2cc(C#N)c(OC)cc12] |
| | 2c |
| | 3-(((2S,3S,4S)-3-ethyl-4-fluoro-5-oxopyrrolidin- |
| | 2-yl)methoxy)-5-methoxybenzo[d]isothiazole- |
| | 6-carbonitrile 2c |
| | 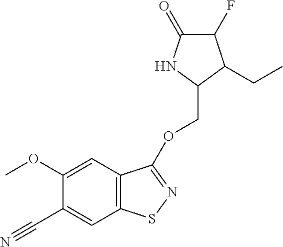 [SMILES: CCC1C(COc2nsc3cc(C#N)c(OC)cc23)NC(=O)C1F] |

In another aspect, this disclosure provides a preparation process of a compound of formula (I) or a pharmaceutically acceptable salt thereof, the preparation process comprising a step of:

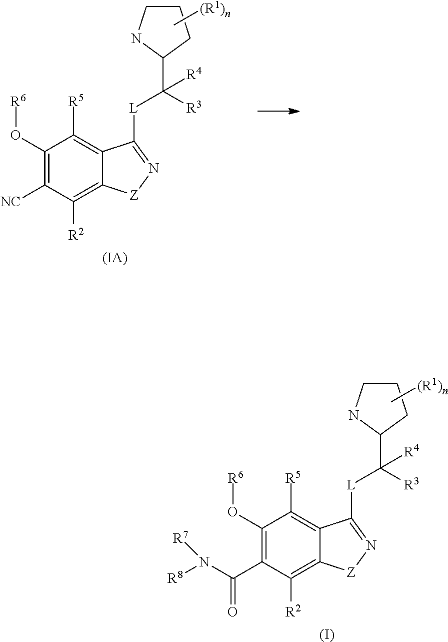

subjecting a compound of formula (IA) to a hydration reaction to obtain the compound of formula (I), wherein:

R⁷ is hydrogen;

R⁸ is hydrogen; and

Z, L, R¹ to R⁶ and n are each as defined in formula (I).

In another aspect, this disclosure provides a preparation process of a compound of formula (II) or a pharmaceutically acceptable salt thereof, the preparation process comprising a step of:

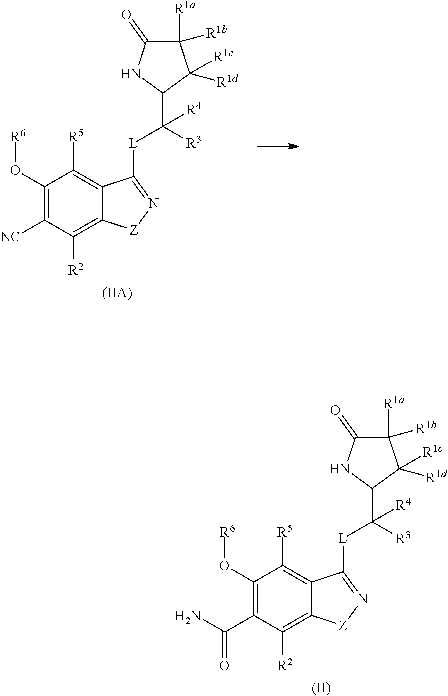

subjecting a compound of formula (IIA) to a hydration reaction to obtain the compound of formula (II), wherein:

Z, L, R²-R⁶ and R^{1a}-R^{1d} are each as defined in formula (II).

In another aspect, this disclosure provides a preparation process of a compound of formula (III) or (III′), or a pharmaceutically acceptable salt thereof, the preparation process comprising a step of:

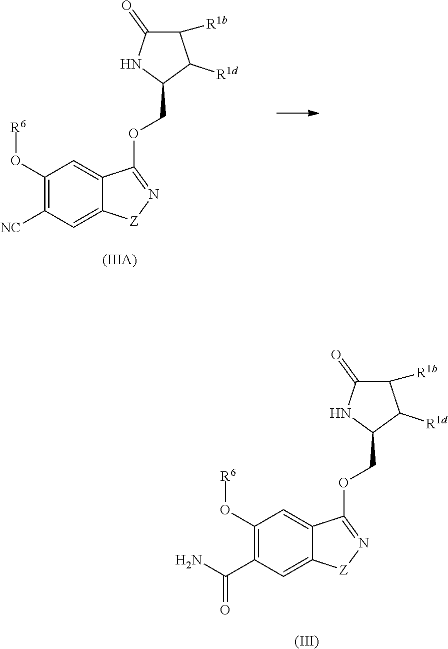

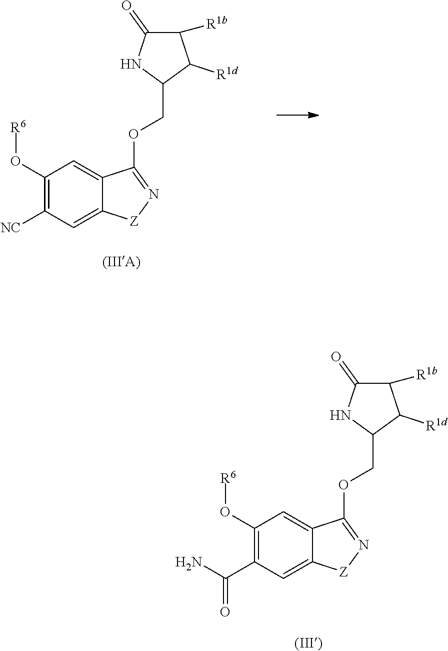

subjecting of a compound of formula (IIIA) to a hydration reaction to obtain the compound of formula (III), or

subjecting of a compound of formula (III′A) to a hydration reaction to obtain the compound of formula (III′), wherein:

Z, R⁶, R^{1b} and R^{1d} are each as defined in formula (III).

The present disclosure also provides a pharmaceutical composition, comprising a compound of formula (I) or a pharmaceutically acceptable salt thereof, and a pharmaceutically acceptable carrier.

The present disclosure also provides a pharmaceutical composition, comprising a therapeutically effective amount of a compound of formula (I) or a pharmaceutically acceptable salt thereof, together with one or more pharmaceutically acceptable carriers, diluents or excipients.

The present disclosure relates to a method of inhibiting IRAK protein kinase, comprising administering to a subject in need thereof a therapeutically effective amount of the compound of formula (I) or a pharmaceutically acceptable salt thereof, or the pharmaceutical composition containing the same; preferably the IRAK protein kinase is an IRAK-4 protein kinase.

The present disclosure relates to a method of preventing and/or treating IRAK-mediated disorder, disease, or condition, comprising a step of administering to a subject in need thereof a therapeutically effective amount of the compound of formula (I) or a pharmaceutically acceptable salt thereof thereof, or pharmaceutical composition containing the same.

The present disclosure relates to a method of preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder, comprising a step of administering to a subject in need thereof a therapeutically effective amount of the compound of formula (I) or a pharmaceutically acceptable salt thereof thereof, or pharmaceutical composition containing the same.

In other words, the present disclosure also relates to use of a compound of formula (I) or a pharmaceutically acceptable salt thereof, or pharmaceutical composition containing the same, in the preparation of a medicament for the inhibition of IRAK protein kinase; preferably the IRAK protein kinase is an IRAK-4 protein kinase.

In other words, the present disclosure also relates to use of a compound of formula (I) or a pharmaceutically acceptable salt thereof, or pharmaceutical composition containing the same, in the preparation of a medicament for preventing and/or treating IRAK-mediated disorder, disease, or condition.

In other words, the present disclosure also relates to use of a compound of formula (I) or a pharmaceutically acceptable salt thereof thereof, or pharmaceutical composition containing the same, in the preparation of a medicament for preventing and/or treating a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The present disclosure further relates to the compound of formula (I) or a pharmaceutically acceptable salt thereof, or pharmaceutical composition containing the same, for use as a medicament.

The present disclosure also relates to the compound of formula (I) or a pharmaceutically acceptable salt thereof, or pharmaceutical composition containing the same, for use in inhibiting IRAK protein kinase; preferably the IRAK protein kinase is an IRAK-4 protein kinase.

The present disclosure also relates to the combination of the compound of formula (I) or a pharmaceutically acceptable salt thereof, or pharmaceutical composition containing the same, for use in preventing and/or treating IRAK-mediated disorder, disease, or condition.

The present disclosure also relates to the combination of the compound of formula (I) or a pharmaceutically acceptable salt thereof, or pharmaceutical composition containing the same, for use in preventing and/or treating a cancer, a neurodegenerative disorder, a viral disease, ail autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

The “cancer” or “proliferative disorder” mentioned includes a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma).

The “MyD88 driven disorder” mentioned includes ABC DLBCL, Waldenstrom's macroglobulmemia, Hodgkin's lymphoma, primary cutaneous T-cell lymphoma and chronic lymphocytic leukemia; and/or the IL-1 driven disorder is Smoldering of indolent multiple myeloma.

The “IL-1” driven disorder mentioned is Smoldering of indolent multiple myeloma.

The “neurodegenerative disease” mentioned includes Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

The “inflammatory disorder” refers to the group consisting of conditions of the eye such as ocular allergy, conjunctivitis, keratoconjunctivitis sicca, and vernal conjunctivitis; diseases affecting the nose including allergic rhinitis; and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, nephritis, vasculitis, diverticulitis, interstitial cystitis, glomerulonephritis (with and without nephrotic syndrome, e.g. including idiopathic nephrotic syndrome or urinal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation; hyperchlolesterolemia, heart disease, chronic heart failure, mesothelioma, anhidrotic ectodermal dysplasia, Behcet's disease, incontinentia pigmenti, Paget's disease, pancreatitis, hereditary periodic fever syndrome; asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis; Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft, rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis; stomatitis, synovitis, tendonitis, tonsillitis, ulcerative colitis, uveitis, vaginitis, vasculitis, vulvitis, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

Common formulations include a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., di chlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or the type of pharmaceutically acceptable salt thereof can be verified by traditional therapeutic regimens.

Unless otherwise stated, the terms used in the specification and claims have the meanings described below.

“Alkyl” refers to a saturated aliphatic hydrocarbon group including C₁-C₂₀ straight chain and branched chain groups. Preferably an alkyl group is an alkyl having 1 to 12 (for example 1, 2, 3, 4, 5, 6, 7, 8, 9, 10, 11 or 12) carbon atoms. Representative examples include, but are not limited to methyl, ethyl, n-propyl, isopropyl, n-butyl, isobutyl, tert-butyl, sec-butyl, n-pentyl. 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-di m ethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 (for example 1, 2, 3, 4, 5 or 6) carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent groups) is one or more, sometimes preferably one to five, and sometimes more preferably one to three, groups independently selected from alkyl, halogen, alkoxy, haloalkoxy, alkenyl, alkynyl, alkylsulfo, alkylamino, thiol, hydroxy, nitro, cyano, amino, cycloalkyl, heterocyclyl, aryl, heteroaryl, cycloalkoxyl, heterocyclyloxy, cycloalkylthio, heterocyclylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, and sometimes more preferably one to three, group(s) independently selected from alkyl, halogen, alkoxy, haloalkoxy, alkenyl, alkynyl, alkylsulfo, alkylamino, thiol, hydroxy, nitro, cyano; amino, cycloalkyl, heterocyclyl, aryl, heteroaryl, cycloalkoxyl, heterocyclyloxy, cycloalkylthio, heterocyclylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc.; preferably C₂₋₂₀ alkynyl, more preferably C₂₋₁₂ alkynyl, and most preferably C₂₋₆ alkynyl. The alkynyl group can be substituted or unsubstituted. When substituted, the substituent groups) is preferably one or more, sometimes preferably one to five, and sometimes more preferably one to three, group(s) independently selected from alkyl, alkenyl, alkynyl, alkoxy, haloalkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 (for example 1, 2, 3, 4, 5, 6, 7, 8, 9, 10, 11 or 12) carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—), etc. The alkylene group can be substituted or unsubstituted. When substituted, the substituent groups) is preferably one or more, sometimes preferably one to five, and sometimes more preferably one to three, group(s) independently selected from alkyl, alkenyl, alkynyl, alkoxy, haloalkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably one to five, and sometimes more preferably one to three, group(s) independently selected from alkyl, alkenyl, to alkynyl, alkoxy, haloalkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Cycloalkyl” refers to a saturated and/or partially unsaturated monocyclic or polycyclic hydrocarbon group having 3 to 20 carbon atoms, preferably 3 to 12 carbon atoms, more is preferably 3 to 10 carbon atoms, and most preferably 3 to 8 (for example 3, 4, 5, 6, 7 or 8) carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclobexadienyi, cyclobeptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a Spiro atom), wherein one or more rings can contain one or more, preferably one to three, double bonds. Preferably a spiro cycloalkyl is 6 to 14 membered, and more preferably 7 to 10 (for example 7, 8, 9 or 10) membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of Spiro cycloalkyl include, but are not limited to the following groups:

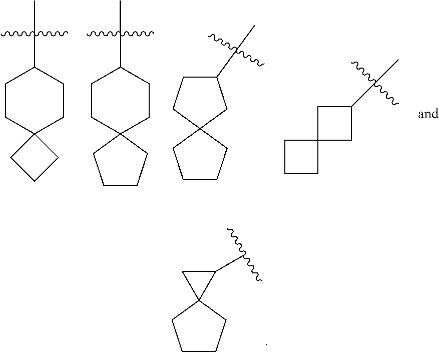

“Fused Cycloalkyl” refers to a 5 to 20 membered polycyclic hydrocarbon group, wherein each ring in the system shares an adjacent pair of carbon atoms with another ring, wherein one or more rings can contain one or more, preferably one to three, double bonds. Preferably, a fused cycloalkyl group is 6 to 14 membered, more preferably 7 to 10 (for example 7, 8, 9 or 10) membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 3-membered/4-membered, 3-membered/5-membered, 3-membered/6-membered, 4-membered/4-membered, 4-membered/5-membered, 4-membered/6-5-membered/4-membered, 5-membered/5-membered, 5-membered/6-membered, 6-membered/3-membered, 6-membered/4-membered, 6-membered/5-membered or 6-membered/6-membered bicyclic fused cycloalkyl. Representative examples of fused cycloalkyls include, but are not limited to, the following groups:

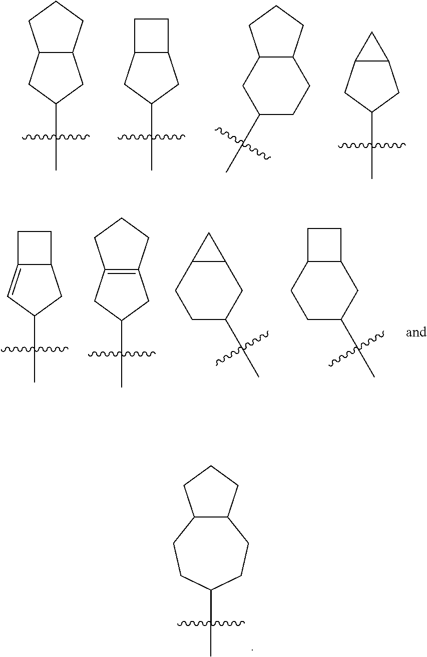

“Bridged Cycloalkyl” refers to a 5 to 2.0 membered polycyclic hydrocarbon group, wherein every two rings in the system share two disconnected carbon atoms. The rings can have one or more, preferably one to three, double bonds. Preferably, a bridged cycloalkyl is 6 to 14 membered, and more preferably 7 to 10 (for example 7, 8, 9 or 10) membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

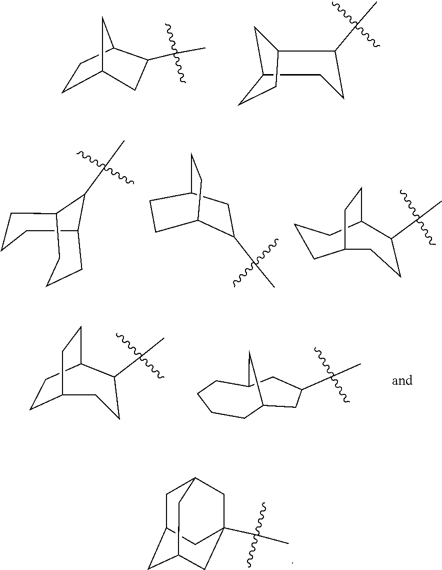

The cycloalkyl include the cycloalkyl said above which fused to the ring of an aryl, heteroaryl or heterocyclyl, wherein the ring bound to the parent structure is cycloalkyl. Representative examples include, but are not limited to indanylacetic, tetrahydronaphthalene, benzocycloheptyl and so on.

The cycloalkyl is optionally substituted or unsubstituted. When substituted, the substituent group(s) is preferably one or more, sometimes preferably one to five, and sometimes more preferably one to three, groups independently selected from alkyl, halogen, alkoxy, haloalkoxy, alkenyl, alkynyl, alkylsulfo, alkylamino, thiol, hydroxy, nitro, cyano, amino, cycloalkyl, heterocyclyl, aryl, heteroaryl, cycloalkoxyl, heterocyclyloxy, cycloalkylthio, heterocyclylthio and oxo group.

“Heterocyclyl” refers to a 3 to 20 membered saturated and/or partially unsaturated monocyclic or polycyclic hydrocarbon group having one or more, sometimes preferably one to five, and sometimes more preferably one to three, heteroatoms selected from N, O, S, S(O) and S(O)₂ as ring atoms, but excluding —O—O—, —O—S— or —S—S— in the ring, the remaining ring atoms being C. Preferably, heterocyclyl is a 3 to 12 (for example 3, 4, 5, 6, 7, 8, 9, 10, 11 or 12) membered having 1 to 4 (for example 1, 2, 3 or 4) heteroatoms; more preferably a 3 to 8 (for example 3, 4, 5, 6, 7 or 8) membered having 1 to 3 (for example 1, 2 or 3) heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfa-morpholinyl, homopiperazinyl, and so on. Polycyclic heterocyclyl includes the heterocyclyl having a Spiro ring, fused ring or bridged ring.

“Spiro heterocyclyl” refers to a 5 to 20 membered polycyclic heterocyclyl with rings connected through one common carbon atom (called a spino atom), wherein said rings have one or more, sometimes preferably one to five, and sometimes more preferably one to three, heteroatoms selected from N, O, S, S(O) and S(O)₂ as ring atoms, the remaining ring atoms being C, wherein one or more rings can contain one or more, preferably one to three, double bonds. Preferably a Spiro heterocyclyl is 6 to 14 membered, and more preferably 7 to 10 (for example 7, 8, 9 or 10) membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of Spiro heterocyclyl include, but are not limited to the following groups:

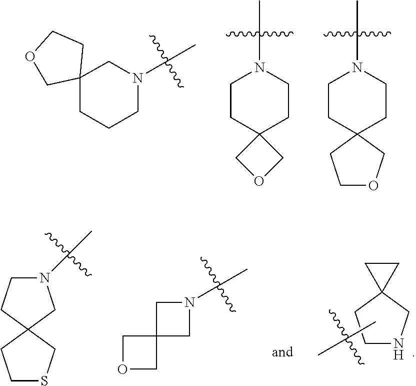

“Fused Heterocyclyl” refers to a 5 to 20 membered polycyclic heterocyclyl group, wherein each ring in the system shares an adjacent pair of carbon atoms with the other ring, wherein one or more rings can contain one or more, preferably one to three, double bonds, and wherein said rings have one or more heteroatoms selected from N, O, S, S(O) and S(O)₂ as ring atoms, the remaining ring atoms being C. Preferably a fused heterocyclyl is 6 to 14 membered, and more preferably 7 to 10 (for example 7, 8, 9 or 10) membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused heterocyclyl. Representative examples of fused heterocyclyl include, but are not limited to, the following groups:

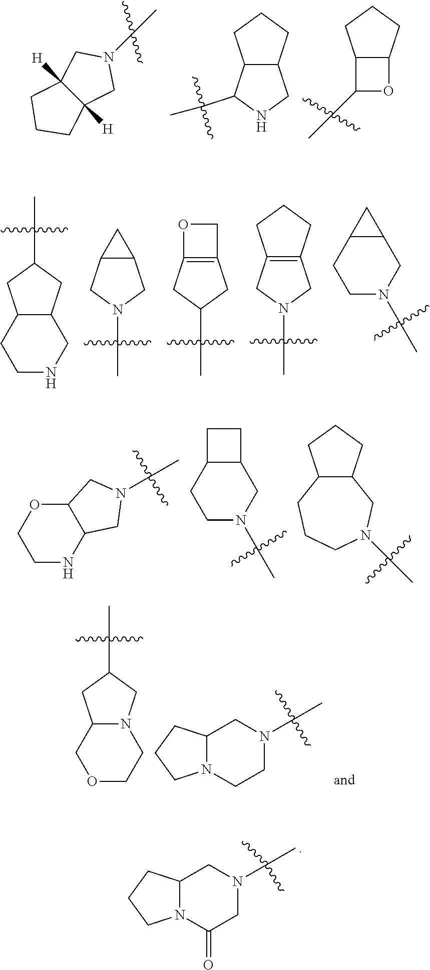

“Bridged Heterocyclyl” refers to a 5 to 14 membered polycyclic heterocyclyl group, wherein every two rings in the system share two disconnected atoms, the rings can have one or more, preferably one to three, double bonds, and the rings have one or more, sometimes preferably one to five, and sometimes more preferably one to three, heteroatoms selected from N, O, S, S(O) and S(O)₂ as ring atoms, the remaining ring atoms being C. Preferably a bridged heterocyclyl is 6 to 14 membered, and more preferably 7 to 10 (for example 7, 8, 9 or 10) membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

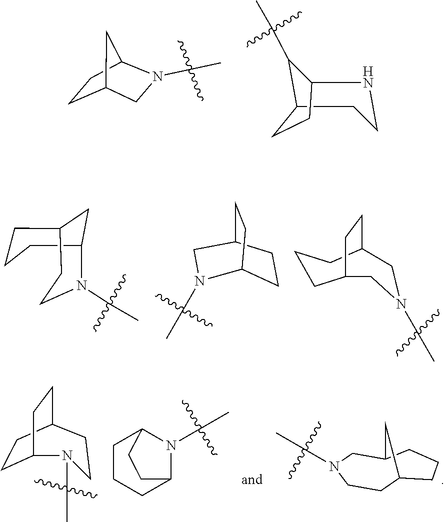

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

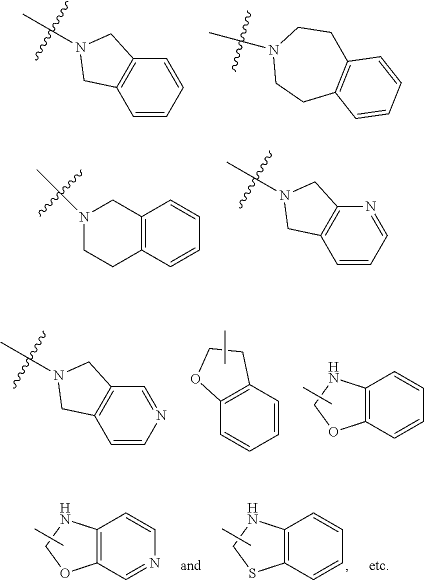

The heterocyclyl is optionally substituted or unsubstituted. When substituted, the substituent group(s) is preferably one or more, sometimes preferably one to five, and sometimes more preferably one to three, group(s) independently selected from alkyl, alkenyl, alkynyl, alkoxy, haloalkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl include the aryl said above which fused to the ring of heteroaryl, heterocyclyl or cycloalkyl, wherein the ring bound to parent structure is aryl. Representative examples include, but are not limited to, the following groups:

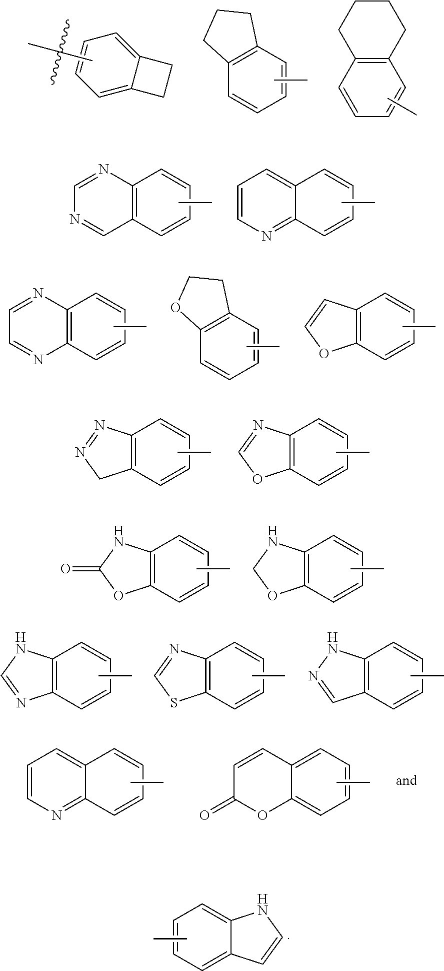

The aryl group can be substituted or unsubstituted. When substituted, the substituent group(s) is preferably one or more, sometimes preferably one to five, and sometimes more preferably one to three, groups independently selected from alkyl, alkenyl, alkynyl, alkoxy, haloalkoxy, alkylsulfo, alkylamino; halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Heteroaryl” refers to an aryl system having 1 to 4 (for example 1, 2, 3 or 4) heteroatoms selected from O, S and N as ring atoms and having 5 to 14 annular atoms. Preferably a heteroaryl is 5- to 10-(for example 5, 6, 7, 8, 9 or 10) membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

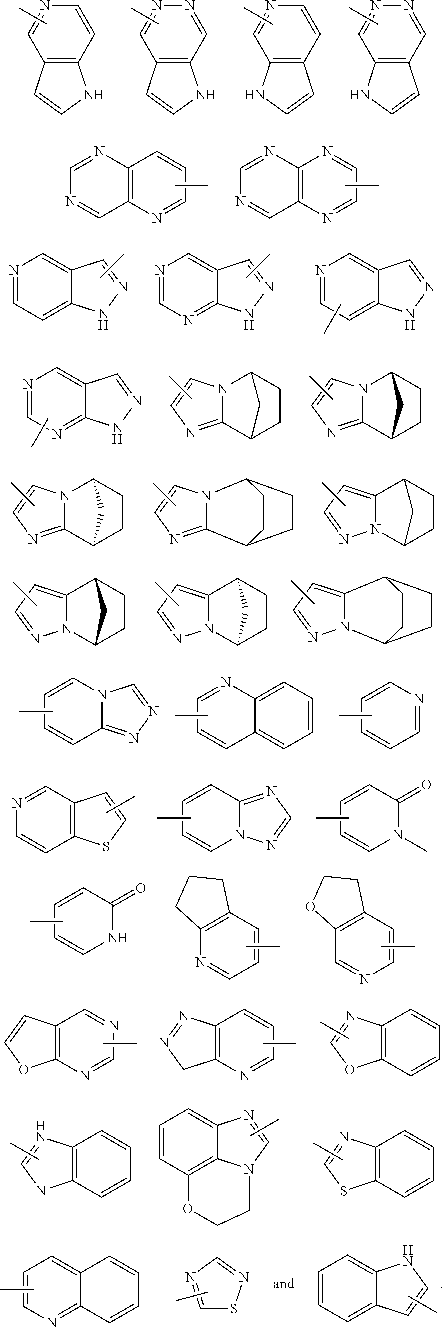

The heteroaryl group can be substituted or unsubstituted. When substituted, the substituent group(s) is preferably one or more, sometimes preferably one to five, and sometimes more preferably one to three, groups independently selected from alkyl, alkenyl, alkynyl, alkoxy, haloalkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent, is preferably one or more, sometimes preferably one to five, and sometimes more preferably one to three, groups independently selected from alkyl, alkenyl, alkynyl, alkoxy, haloalkoxy, alkyl sulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

The above-mentioned cycloalkyl, heterocyclic, aryl and heteroaryl groups contain one monovalent residue derived from the removal of one hydrogen atom from the parent ring atom, or one divalent residue derived from the removal of two hydrogen atoms from the same or different ring atoms of the parent, namely “divalent cycloalkyl”, “divalent heterocyclic group”, “arylene”, and “heteroarylene”.

The term “amino protecting group” is to keep the amino group unchanged when other parts of the molecule react, and to protect the amino group with a group that is easy to remove. Non-limiting examples include (trimethylsilyl)ethoxymethyl, tetrahydropyranyl, t-butoxycarbonyl, acetyl, benzyl, allyl, p-methoxybenzyl, and the like, sometimes preferably tetrahydropyranyl and further preferably tetrahydropyran-2-yl. These groups may be optionally substituted with 1-3 substituents selected from halogen, alkoxy or nitro.

“Bond” refers to a covalent bond using a sign of “—”.

“Hydroxyalkyl” refers to an alkyl group substituted by one or more, preferably one to three, sometimes preferably one, hydroxy groups, wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(═O)OH group.

“Haloalkyl” refers to an alkyl group substituted by one or more halogen groups, wherein alkyl is as defined above.

“Haloalkoxy” refers to a haloalkyl-O—, wherein alkyl is as defined above.

“Thiol” refers to a —SH group.

“Alkylthio” refers to an alkyl-S— group, wherein alkyl is as defined above.

“Haloalkylthio” refers to a haloalkyl-S— group, wherein haloalkyl is as defined above.

“Cycloalkoxyl” refers to a cycloalkyl-O—, wherein cycloalkyl is as defined above.

“Heterocyclyloxy” refers to a heterocyclyl-O—, wherein heterocyclyl is as defined above.

“Cycloalkylthio” refers to a cycloalkyl-S—, wherein cycloalkyl is as defined above.

“Heterocyclylthio” refers to a heterocyclyl-S—, wherein heterocyclyl is as defined above.

THP refers to

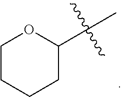 [SMILES: *C1CCCCO1]

Boc refers to

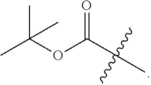 [SMILES: *C(=O)OC(C)(C)C]

OTf refers to

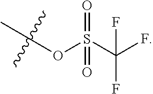 [SMILES: *OS(=O)(=O)C(F)(F)F]

“Alkoxycabonyl” refers to a —C(═O)O(alkyl), C(═O)O (cycloalkyl), (alkyl)C(O)O— or (cycloalkyl)C(O)O— group, wherein the alkyl and cycloalkyl are defined as above.

“Optional” or “optionally” means that the event or circumstance described subsequently can, but need not, occur, and the description includes the instances in which the event or circumstance may or may not occur. For example. “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

In some embodiments, when an alkyl, alkenyl, alkynyl, cycloalkyl, heterocyclyl, aryl, heteroaryl group, or the like, or a moiety thereof, is substituted, the substituents, sometimes preferably 1 to 5, and sometimes more preferably 1 to 3, are preferably independently selected from C₁-C₆ alkyl, halogen, C₁-C₆ alkoxy, C₁-C₆ alkenyl, C₁-C₆ alkynyl, C₁-C₆ alkylthio. C₁-C₆ alkylamino, alkylamino, thiol, hydroxyl, nitro, cyano, amino, C₃-C₆ cycloalkyl, 5- to 10-membered heterocyclyl, C₆-C₁₀ aryl, 5- to 10-membered heteroaryl, C₃-C₆ cycloalkoxy, C₁-C₆ cycloalkylthio, 5- to 10-membered heterocyclylthio and oxo group. In some embodiments, sometimes more preferably, the substituents are independently selected from C₁-C₄ alkyl, halogen, alkoxy, alkylthio, alkylamino; alkylamino, thiol, hydroxyl, nitro, cyano, and amino. As a person of ordinary skill in the art would understand, an oxo (═O) group cannot be a substituent on an unsaturated carbon, such as those in the ring of an aryl or heteroaryl group.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C); or non-radioactive isotopes, such as deuterium (D) or carbon-13 (¹³C). Such isotopic varitations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

### Synthesis Method

In order to complete the purpose of the disclosure, the present disclosure applies, but is not limited to, the following technical solution:

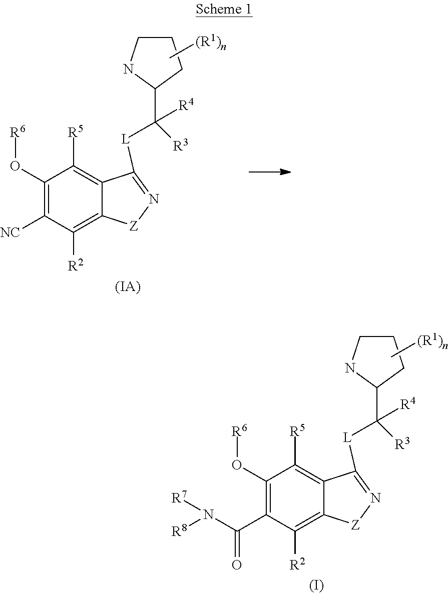

A preparation process of a compound of formula (I) or a pharmaceutical acceptable salt thereof comprising a step of:

the compound of formula (IA) is subjected to hydration reaction under alkaline conditions in the presence of catalyst, to obtain the compound of formula (I), wherein:

R⁷ is hydrogen;

R⁸ is hydrogen;

Z, L, R¹ to R⁶ and n are each as defined in formula (I),

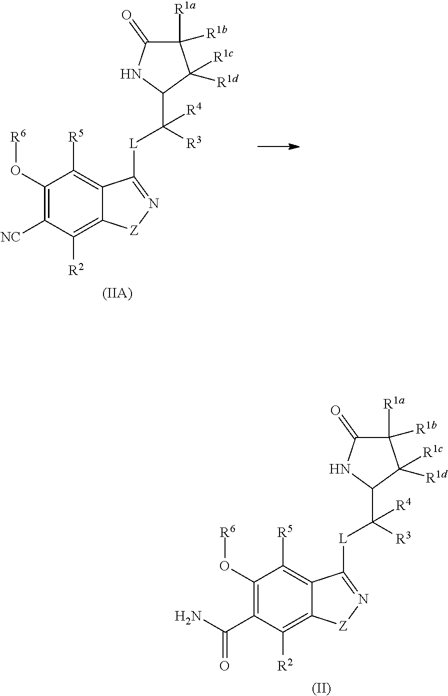

A preparation process of a compound of formula (II) or a pharmaceutically acceptable salt thereof, thereof comprising a step of:

the compound of formula (IIA) is subjected to hydration reaction under alkaline conditions in the presence of catalyst, to obtain the compound of formula (II), wherein:

Z, L, R²-R⁶ and R^{1a}-R^{1d} are each as defined in formula (II).

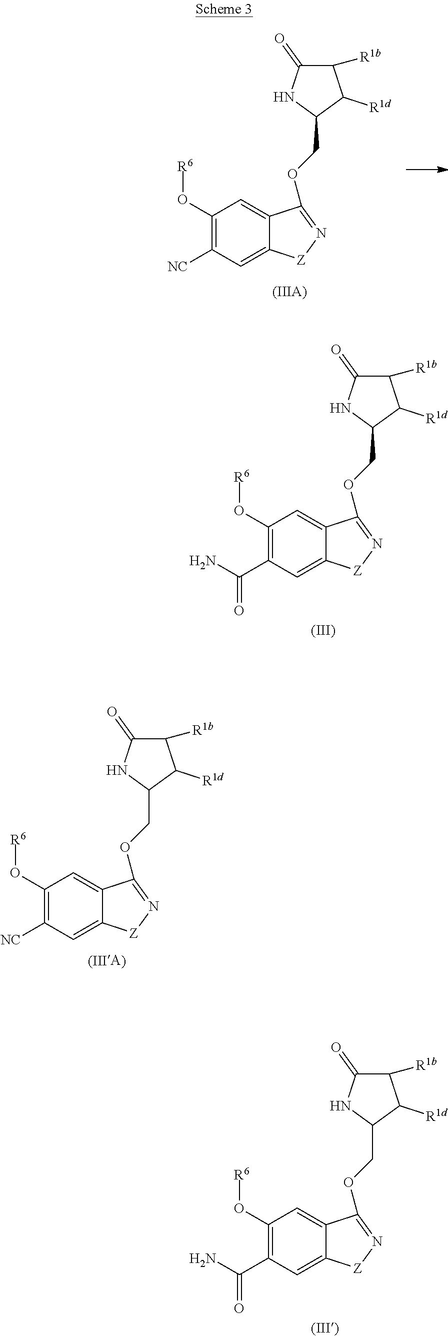

A preparation process of a compound of formula (III) or a pharmaceutically acceptable salt thereof comprising a step of:

the compound of formula (IIIA) is subjected to hydration reaction under alkaline conditions in the presence of catalyst, to obtain the compound of formula (III), or

the compound of formula (III′A) is subjected to hydration reaction under alkaline conditions in the presence of catalyst, to obtain the compound of formula (III′),

wherein:

Z, R⁶, R^{1b} and R^{1d} are each as defined in formula (III).

The reagents that provide alkaline conditions include organic bases and inorganic bases, wherein the organic base includes, but is not limited to, triethyl amine, diethylamine, N,N-disopropylethylamine, n-butyllithium, lithium diisopropyl amide, potassium acetate, sodium tert-butoxide, N-(3-Dimethylaminopropyl)-N′-ethylcarbodiimide hydrochloride (EDCI), potassium bis(trimethylsilyl)amide (KHMDS) and potassium Cert-butoxide, and wherein the inorganic base includes, but is not limited to, magnesium chloride, sodium hydride, potassium phosphate, sodium carbonate, potassium carbonate, cesium carbonate and sodium hydroxide; preferably potassium carbonate.

The catalysts for the hydrolysis reaction include, but are not limited to, MnO₂, H₂O₂, NiO, CeO₂ and ruthenium hydroxide; preferably is H₂O₂.

The reaction is preferably conducted in solvent, wherein suitable solvents include, but are not limited to, acetic acid, methanol, ethanol, ether, toluene, tetrahydrofuran, dichloromethane, dimethylsulfoxide, 1,4-dioxane, water, N,N-dimethylformamide and the mixture thereof.

### Methods of Preparation

### General Synthetic Schemes

The compounds of the present invention can be prepared in several ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. The reactions and techniques described in this section are performed in solvents appropriate to the reagents and materials employed and are suitable for the transformations being affected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene et al. (Protective Groups in Organic Synthesis, Third Edition, Wiley and Sons (1999)). Compounds of the Formula (I) can be prepared according to the methods outlined in the following schemes.

### Experimental Procedures and Working Examples

The following illustrate the synthesis of various compounds of the present invention. Additional compounds within the scope of this invention may be prepared using the methods illustrated in these Examples, either alone or in combination with techniques generally known in the art. It will be understood that the intermediate compounds of the invention depicted above are not limited to the particular enantiomer shown, but also include all stereoisomers and mixtures thereof. It will also be understood that compounds of Formula (I) can include intermediates of compounds of Formula (I).

### Experimental Procedures

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography and/or liquid chromatography mass spectrometry, and subjected to work-up when appropriate. It will be recognized by one skilled in the art that purifications may vary between experiments: in general, sorbents, solvents and the solvent ratios used for eluants/gradients were chosen to provide appropriate Rf s or retention times. It will also be recognized by one skilled in the art that MIX purifications may be effected in a variety of ways, including the use of normal stationary phases, reverse stationary phases, chiral stationary phases, and supercritical eluants. The appropriate choices of conditions for chromatographic and HPLC purifications will be discerned by one skilled in the art. The following Preparations describe the preparation of certain intermediates used in the Methods and Examples that follow. All patent or non-patent references cited in this application are incorporated by reference in their entirety for all purpose without admission of them as prior art.

### General Information

The structure of the compound was determined by nuclear magnetic resonance (NMR)/mass spectrometry (MS). All NMR spectra were recorded on Bruker AVANCE II-400 MHz spectrometer at STP unless otherwise indicated. NMR chemical shifts were reported in ppm and referenced to TMS (δ=0.00 ppm, ¹H NMR) or the residual solvent peak, Deuterated solvents such as DMSO-d⁶, CDCl₃ and CD₃OD were used in general NMR test.

LC/MS (ESI) analyses were performed on a Shimadzu LCMS2020 equipped with a Sunfire C18 (5 μm 50×4.6 mm) column, Waters UPLC-QDa equipped with an ACQUITY UPLC® BEH (2.1*50 mm 1.7 um) column, Agilent Agilent6120 equipped with a Xbridge C₁₈ (5 μm 50×4.6 mm) column.

HPLC analyses were performed on an Agilent 1200DAD equipped with a Sunfire CIS (5 μm 150×4.6 mm) column and Shimadzu UFLC equipped with an Xbridge C18 (5 μm 150×4.6 mm) column.

Chiral HPLC analyses were performed on a Waters-UPC² instrument.

Analytical thin layer chromatography (TLC) was performed using 0.2 mm-0.25 mm silica gel plates (purchased from Huanghai Yantai, Xinnuo and Shandong Rushan). Prepared thin layer chromatography (PLC) was performed on using 0.4 mm-0.5 mm silica gel plates.

Column chromatography was performed using silica gel 100-200 mesh or 200-300 mesh (purchased from YuCheng Chemical Shanghai, Co., Ltd) as the solid support.

Pre-HPLC was performed on a Waters 2767 equipped with a Sunfire Pre C18 (10 μm 19×250 mm) column and Waters 2767-QDa equipped with an Xbridge Pre C18 (10 μm 19/250 mm) column instrument.

Pre-SFC was performed on a Waters-SFC80 equipped with Daciel AD/OD/OJ/IC/IA/ID (10 μm 20×250 mm) column instrument.

CombiFlash was performed on an Agela Technologies.

The source should be indicated if the starting material was purchased and used as received. For example: ABCR GmbH & Co. KG, Acros Organics, Aldrich Chemical Company, Accela ChemiBio Inc, etc.

All reactions were carried out under an atmosphere of nitrogen unless otherwise indicated.

High pressure hydrogenation was performed on GSH-1/12.5, GSH-2/12.5 GSH-5/12.5. GSH-20/12.5 autoclave.

Microwave reaction was performed on a Monowave 300 and Initiateor+.

The ratio of eluent system should be recorded clearly when using TLC or purification with silica gel chromatography column. The addition of small amounts of TEA or acetic acid and other basic or acidic reagents also needs to be clearly labeled.

The following Preparations, Methods and Examples are intended to illustrate particular embodiments of the invention and preparations thereto and are not intended to limit the specification, including the claims, in any manner. Unless noted otherwise, all reactants were obtained commercially.

Unless indicated otherwise, the following abbreviations have the indicated meanings:
APCI—atmospheric pressure chemical ionization
br.—broad peaks
° C.—degree Celsius
CDCl₃—deuterated chloroform
CD₃OD—deuterated methanol
d—doublet peak
dd—double doublet peak
D₂O—deuterium oxide
dmso-ds—perdeuterated dimethyl sulfoxide
dt—double triplet peak
g—gram(s)
H (e.g., 1H, 2H)—hydrogen(s)
hr—hour(s)
LC—liquid chromatography
m—multiplet
M—molarity
mg—milligram(s)
MHz—megahertz
min—minute(s)
mL—milliliter(s)
mmol—millimole(s)
mp—melting point
MS—mass spectrum
NMR—nuclear magnetic resonance
pH—negative logarithm of hydronium ion concentration
psi—pounds per square inch
q—quartet peak
s—singlet peak
t—triplet peak
td—triple doublet peak
μL—microliter

### Preparations

### Example 1: (S)-5-methoxy-3-((5-oxopyrrolidin-2-yl)methoxy)benzo[d]isothiazole-6-carboxamide 1

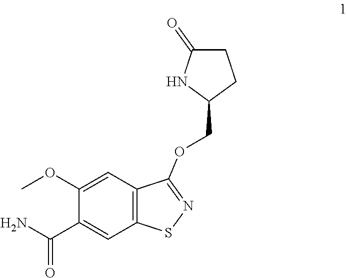 [SMILES: COc1cc2c(OC[C@@H]3CCC(=O)N3)nsc2cc1C(N)=O]

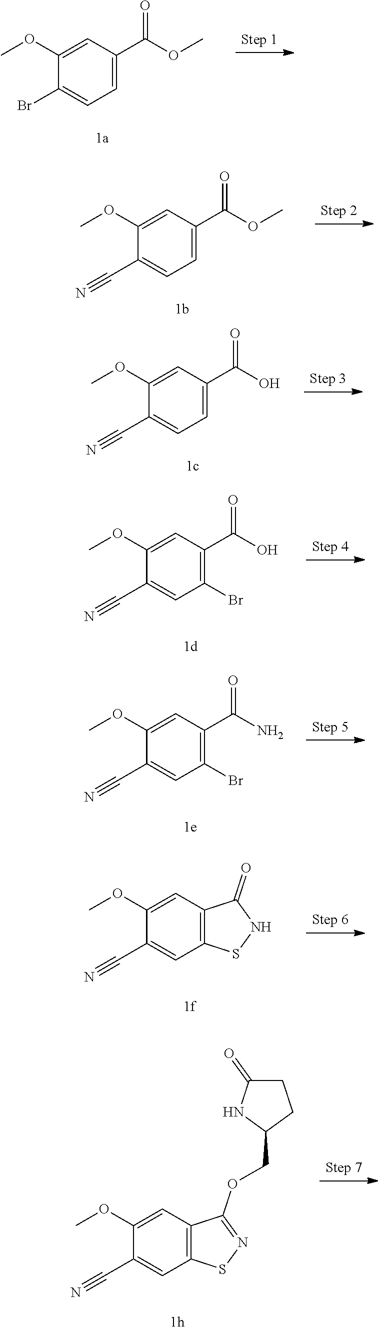

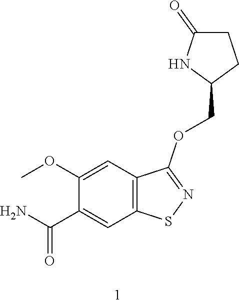 [SMILES: COc1cc2c(OC[C@@H]3CCC(=O)N3)nsc2cc1C(N)=O]

### Step 1. Synthesis of: ethyl 4-cyano-3-methoxy-benzoate 1b

To a solution of methyl 4-bromo-3-methoxybenzoate 1a (7.1 g, 28.97 mmol, Bidepharm) in DMF (40 mL), KI (6.3 g, 37.95 mmol), CuCN (5.3 g, 56.62 mmol) and CuI (6.7 g, 35.18 mmol) were added. The resulting mixture was stirred at 150° C. for 20 h under protection of argon. After the reaction was cooled down to RT, it was filtrated. The filtrate was poured into water (150 mL). Then the mixture was extracted with ethyl acetate (30×3). The organic layers were combined and washed with water and brine, dried over anhydrous Na₂SO₄ and concentrated. The residue was purified by silica gel column chromatograph (petroleum ether/dichloromethane=1/1) to give 1b (5 g, 26.15 mmol, 90.28% yield).

### Step 2. Synthesis of 4-cyano-3-methoxy-benzoic Acid 1e

To a solution of 1b (18 g, 94.15 mmol) in H₂O (200 mL) and THF (200 mL) was added LiOH (4.52 g, 188.3 mmol, bide). The mixture was stirred at room temperature for 1 hour. The pH was adjusted to 3 with aq. HCl. The mixture was extracted with EtOAc. The combined organic layers were washed with brine, dried over Na₂SO₄ and concentrated to give crude 1c (15.7 g, 88.62 mmol, 94.13% yield).

### Step 3. Synthesis of 2-bromo-4-cyano-5-methoxy-benzoic Acid 1d

To a solution of 1c (200 mg, 1.13 mmol) in HOAc (2 mL) and H₂O (2 mL), Br₂ (159.81 g, 1.13 mmol, SCRC) was added and the resulting mixture was stirred at 60° C. for 20 hours. After the mixture was cooled to room temperature, the mixture was diluted with EtOAc and water. The organic layer was separated. The combine organic layer was washed with brine, dried over sodium sulfate, filtered and concentrated to give 1d (260 mg. 1.02 mmol, 89.86% yield).

### Step 4. Synthesis of 2-bromo-4-cyano-5-methoxy-benzamide 1e

To a stirred solution of 1d (190 mg, 742.03 μmol) and DMF (one drop) in DCM (5 mL) at 0° C. was added oxalyl chloride (470.92 mg, 3.71 mmol). The mixture was stirred at rt, for 2 h. The mixture was concentrated under reduced pressure. To the residue was added THF (10 mL) to get a solution. The solution was added to the stirred solution of ammonium hydroxide (2.60 g, 74.20 mmol) at 0° C. slowly. After addition, the mixture was stirred at rt for 2 h. The mixture was extracted with EtOAc (15 mL×2). The combined organic layer was dried, concentrated. The residue was purified by silica gel chromatography (EtOAc) to give 1e (95 mg, 372.45 nmol, 50,19% yield). MS m/z (ESI): 255 [M+1].

### Step 5. Synthesis of 5-methoxy-3-oxo-1,2-benzothiazole-6-carbonitrile 1f

The mixture of 1,10-phenanthroline (2119?6 mg, 1.18 mmol) and CuI (224.00 mg, 1.18 mmol) in DMF (25 mL) was stirred at rt for 30 min under N₂. To the mixture was added 1d (1 g, 3.92 mmol), S₈ (301.71 mg, 4.70 mmol) and potassium carbonate (1.08 g, 7.84 mmol), The mixture was stirred at 110° C. overnight under N₂. The mixture was concentrated under reduced pressure, the residue was diluted with EtOAc (80 mL), and the organic layer was washed with sat. aq. NH₄Cl (30 mL×4), dried and concentrated. The residue was purified by silica gel chromatography (THF) to give 1f (330 mg, 1.60 mmol, 40.82% yield). MS m/z (ESI): 207 [M+1].

### Step 6. Synthesis of (S)-5-methoxy-3-((5-oxopyrrolidin-2-yl)methoxy)benzo[d]isothiazole-6-carbonitrile 1 h

The mixture of 1f (60 mg, 290.95 μmol), (5S)-5-(bromomethyl)pyrrolidin-2-one (54.39 mg, 305.50 μmol, Energy) and potassium carbonate (80.42 mg, 581.90 μmol) in DMF (3 mL) was stirred at 80° C. for 5 h. The mixture was concentrated, and the residue was purified by silica gel chromatography (EtOAc) to give 1h (40 mg, 131.87 μmol, 45.32% yield). MS m/z (ESI): 304 [M+1].

### Step 7. Synthesis of (S)-5-methoxy-3-((5-oxopyrrolidin-2-yl)methoxy)benzo[d]isothiazole-6-carboxamide 1

To a stirred mixture of 1b (40 mg, 131.87 μmol) and potassium carbonate (36.45 mg, 263.73 μmol) in DMSO (3 mL) was added hydrogen peroxide (13.46 mg, 395.60 μmol). The mixture was stirred at rt for 2 h and was quenched with H₂O, the resulting remain was filtered, and the filter cake was triturated with methanol (3 mL×2) to give the title compound 1 (36 mg, 112.03 μmol, 84,95% yield).

NMR (400 MHz, DMSO, ppm): δ 8.31 (s, 1H), 8.12 (s, 1H), 7.84 (s, 1H), 7.69 (s, 1H), 7.53 (s, 1H), 4.55 (dd, 1H), 4.27 (dd, 1H), 4.01 (br, 1H), 3.95 (s, 3H), 2.33-2.13 (m, 3H), 1.90-1.84 (m, 1H). MS m/z (ESI): 322 [M+1].

### Example 2: (3-(((2S,3S,4S)-3-ethyl-4-fluoro-5-oxopyrrolidin-2-yl)methoxy)-5-methoxybenzo[d]isothiazole-6-carboxamide 2

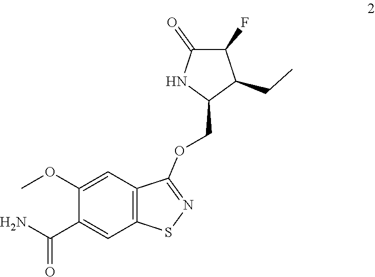 [SMILES: CC[C@@H]1[C@H](F)C(=O)N[C@@H]1COc1nsc2cc(C(N)=O)c(OC)cc12]

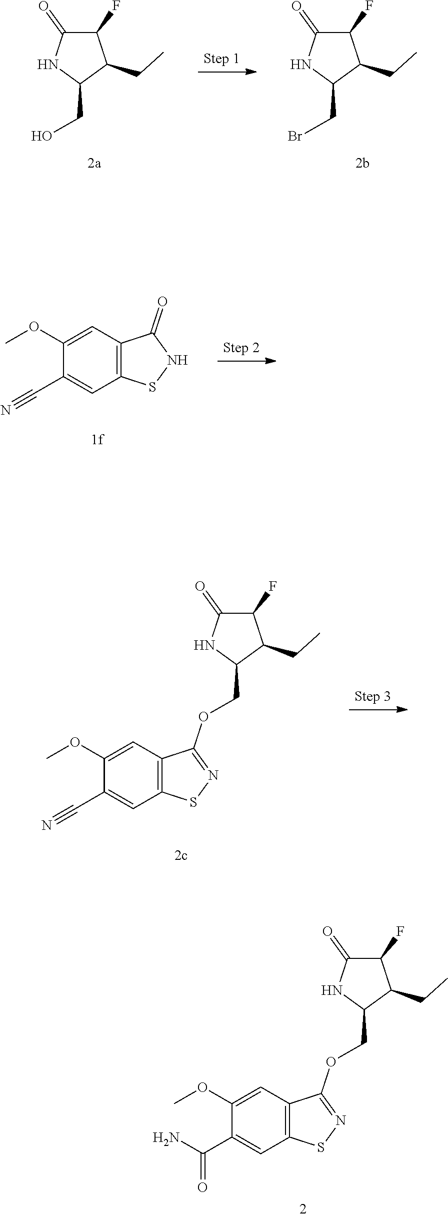

### Step 1

### Step 1. Synthesis of (3S,4S,5S)-5-(bromomethyl)-4-ethyl-3-fluoropyrrolidin-2-one 2b

To a solution (3S,4S,5S)-4-ethyl-3-fluoro-5-(hydroxymethyl)pyrrolidin-2-one 2a (200.00 mg, 1.24 mmol, PharmaBlock) and PPh₃ (358 mg, 1.24 mmol) in acetonitrile (2.6 mL) was added the solution of CBr₄ (453 mg, 1.24 mmol) in acetonitrile (1 mL) drop-wise at 0° C. The resulting clear solution was stirred at rt overnight, The solvent was removed by rotary evaporation and the residue was purified by silica gel chromatography (EA/PE=1:3) to afford crude product 2b (300 mg, 1.34 mmol, 107.90% yield). MS m/z (EST): 226 [M−1].

### Step 2. Synthesis of 3-(((2S,3S,4S)-3-ethyl-4-fluoro-5-oxopyrrolidin-2-yl)methoxy)-5-methoxybenzo[d]isothiazole-6-carbonitrile 2c

The mixture of 1f (50 mg, 242.46 μmol), the compound 2b (59.76 mg, 266.70 μmol) and potassium carbonate (67.02 mg 484.92 μmol) in DMF (5.0 nit) was heated to 80° C. for 2 h. The mixture was concentrated under reduced pressure and the residue was purified by silica gel chromatography (THF) to give 2c (45 mg, 128.80 μmol, 53.12% yield). MS m/z (ESI): 350 [M+1].

### Step 3. Synthesis of 3-(((2S,3S,4S)-3-ethyl-4-fluoro-5-oxopyrrolidin-2-yl)methoxy)-5-methoxybenzo[d]isothiazole-6-carboxamide 2

To a stirred mixture of 2e (45 mg, 128.80 μmol) and potassium carbonate (53.40 mg 386.40 μmol) in DMSO (2 mL) at rt was added hydrogen peroxide (13.14 mg, 386.40 μmol, 30% in water). The mixture was stirred at rt for 3 h. To the mixture was added H₂O (10 mL), The resulting remain was filtered and purified by prep-HPLC to give 2 (20 mg, 54.44 μmol, 42.27% yield. ¹H NMR (400 MHz, DMSO, ppm): δ 8.93 (s, 1H), 8.31 (s, 1H), 7.84 (s, 1H), 7.69 (s, 1H), 7.57 (s, 1H), 4.91 (dd, 1H), 4.62 (dd, 1H), 4.23-4.17 (m, 1H), 4.08 (brs, 1H), 3.93 (s, 3H), 2.65-2.61 (m, 1H), 1.59-1.53 (m, 2H), 1.02 (t, 3H). MS m/z (ESI): 368 [M+1].

### Biological Assays

The present disclosure will be further described with reference to the following test examples, but the examples should not be considered as limiting the scope of the disclosure.

### Test Example 1. LRAM, Biochemical Assay

### Experimental Procedure:

For the routine compound screening, an IRAK4 kinase assay was performed as follows using Promega ADP-Glo IRAK4 Kinase Enzyme System (Promega, Cat #2198-AD). Purified full length human IRAK4 protein (Signal Chem; Cat #1112-10G-125) was diluted to a concentration 6 nM in assay buffer (25 mM MOPS, pH 7.2, 12.5 mM β-glycerol-phosphate, 25 mM MgCl2, 5 mM EGTA, 2 mM EDTA, 0.0025% Brij-35, add 0.25 mM DTT to Kinase Assay Buffer prior to use) containing IRAK4 inhibitor at 2× the final concentration in 0.4% DMSO. The reaction was performed in PerkinElmer Proxiplate-384 plus white plate (Fisher Scientific, Cat #50-905-2761) and started by the addition of an equal volume of the assay buffer containing 4 μM native swine myelin basic protein (MBP) and 500 μM ATP to achieve a final concentration of 3 nM enzyme, 2 μM MBP, 250 μM ATP, 1×compound, and 0.2% DMSO. The kinase reaction was allowed to run for 60 min at room temperature (25-27° C.), then 5 μL of ADP-Glo was added to the reaction in each well followed by 40 minutes-incubation at RT. To detect converted ATP, 10 μL Detection Buffer was added into each well and Incubate at RT in dark for 30 minutes. Finally, luminescence was read using Tecan with 1000 ms of integration time. The luminescent signal positively correlates with ADP amount and kinase activity. Data was analyzed by GraphPad Prism and IC50 was calculated using function log(inhibitor) vs. response—Variable slope (four parameters). Percentage of inhibition was calculated as below:

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no IRAK4 kinase.

Data Analysis: The assay results are listed in Table 1.

TABLE 1

| | IRAK4 | IRAK4 Biochemical |
|---|---|---|
| Example | Biochemical Assay | Assay Max |
| No. | (IC₅₀, nM) | Inhibition (%) |
| 1 | 240 | 98.0 |
| 2 | 14.0 | 93.0 |

Conclusion: The compounds of the present disclosure have a significant inhibition effect on the enzymatic activity of IRAK4.

### Test Example 2. IRAK4 Human PBMC TNFα Cell Based Assay

### Experimental Procedure:

In this assay, serially diluted compounds were incubated with 1×10⁴ human peripheral blood mononuclear cells (PBMCs) from STEMCELL (Cat #70025.1) per well of 384-well plate (Fisher Scientific; 50-905-2761) and cultured with TexMACS medium (Miltenyi, Cat #130-097-196), stimulated by R848 (0.5 μM) or IL-1β (10 ng/ml). Plates were covered with lids and incubated for 4 h (R848, Fisher Scientific, Cat #NC9801605) or 24 h (IL-1β, Fisher Scientific, Cat #ENRIL1BI) at 37° C. in a humidified tissue-culture incubator. After a brief centrifugation at 1000 rpm for 5 minutes, 5 μl of culture medium was used for the measurement of the TNFα amount using TNFα (human) AlphaLISA Detection Kit (PerkinElmer, Cat #AL208C) according to manufacturer's protocol. A biotinylated anti-TNFα antibody binds to the Streptavidin-coated Donor beads while another anti-TNFα antibody is conjugated to AlphaLISA Acceptor beads. In the presence of the TNFα, the beads come into close proximity. The excitation of the Donor beads causes the release of singlet oxygen molecules that triggers a cascade of energy transfer in the Acceptor beads, resulting in a sharp peak of light emission at 615 nm. AlphaLISA assay plates were read using PHERAstar. Percentage of inhibition was calculated as below:

% inhibition=(1−(Reading(Sample)−Reading(Negative Control))/(Reading(Positive Control)−Reading(Negative Control)))×100%

The 0% inhibition value comes from the Reading of Positive Control wells having 0 nM compound. The 100% inhibition value comes from the Reading of Negative Control wells having no R848 or IL-1β.

Data Analysis: The assay results are listed in Table 2.

TABLE 2

| | IRAK4 PBMC | IRAK4 PBMC |
|---|---|---|
| | AlphaLISA R848 | AlphaLISA IL-1β |
| Example No. | (IC₅₀, nM) | (IC₅₀, nM) |
| 2 | 21.0 | 26.0 |

Conclusion: The compounds of the present disclosure have a significant inhibition effect on the cellular activity of IRAK4.

What is claimed is:

1. A compound of formula (I) or a pharmaceutically acceptable salt thereof: 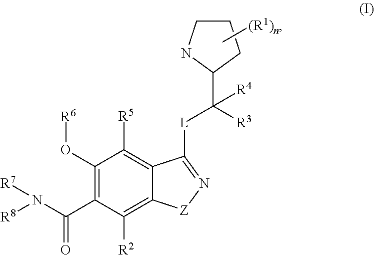
wherein:
Z is O or S;
L is O, S(O)ⱼ, CR^{c}R^{d} or NRⁿ;
R¹ at each occurrence is independently selected from hydrogen, halogen, alkyl, haloalkyl, alkoxy, alkylthio, haloalkoxy, haloalkylthio, hydroxy, hydroxyalkyl, cyano, amino, —(CH₃)ᵣ—NR^{a}R^{b}, nitro, oxo, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more groups independently selected from halogen, alkyl, haloalkyl, alkoxy, alkylthio, haloalkoxy, haloalkylthio, hydroxy, hydroxy alkyl, cyano, amino, nitro, —(CH₂)ₛ—NR^{e}R^{f} and cycloalkyl;
or two R¹ together with the attached atoms form a cycloalkyl or heterocyclyl; wherein the cycloalkyl and heterocyclyl are optionally substituted with one or more groups independently selected from halogen, alkyl, haloalkyl, alkoxy, alkylthio, haloalkoxy, haloalkylthio, hydroxy, hydroxyalkyl, cyano, amino, nitro and —(CH₂)ₛ—NR^{e}R^{f};
R² and R⁵ are identical or different and are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b}, nitro, cycloalkyl, heterocyclyl, aryl and heteroaryl; wherein the alkyl, cycloalkyl, heterocyclyl, aryl or heteroaryl is optionally substituted with one or more groups independently selected from halogen, alkyl, haloalkyl, alkoxy, haloalkoxy, hydroxy, hydroxyalkyl, cyano, amino, nitro, —(CH₂)ₛ—NR^{e}R^{f}, cycloalkyl, heterocyclyl, aryl and heteroaryl;
R³, R⁴, R^{c} and R^{d} are identical or different and are independently selected from hydrogen, halogen, alkyl, haloalkyl, alkoxy, alkylthio, haloalkoxy, haloalkylthio, hydroxy, hydroxyalkyl, cyano, amino and —(CH₂)ᵣ—NR^{a}R^{b};
R⁶ is selected from alkyl, alkenyl, alkynyl, cycloalkyl, heterocyclyl, aryl and heteroaryl; wherein the alkyl, alkenyl, alkynyl, cycloalkyl, heterocyclyl, aryl and heteroaryl are optionally substituted with one or more groups independently selected from halogen, alkyl, haloalkyl, alkoxy, alkylthio, haloalkoxy, hydroxy, hydroxyalkyl, cyano, amino, nitro, —(CH₂)ₛ—NR^{e}R^{f} and cycloalkyl;
R⁷, R⁸, R^{a}, R^{b}, R^{e} and R^{f} are identical or different and at each occurrence are independently selected from hydrogen, alkyl, haloalkyl, hydroxyalkyl, —C(═O)OR^{q}, cycloalkyl, heterocyclyl, aryl and heteroaryl;
or R⁷ and R⁸, R^{a} and R^{b}, R^{e} and R^{f} together with the nitrogen to which they are attached form a heterocyclyl; wherein the heterocyclyl is optionally substituted with one or more groups independently selected from halogen, alkyl, haloalkyl, alkoxy, haloalkoxy, hydroxy, hydroxyalkyl, oxo, cyano, amino, nitro, cycloalkyl, heterocyclyl, aryl and heteroaryl;
Rⁿ is selected from hydrogen, alkyl, haloalkyl, hydroxyalkyl, cycloalkyl, heterocyclyl, aryl and heteroaryl;
R^{q} is selected from hydrogen, alkyl and haloalkyl;
n is 0, 1, 2, 3 or 4;
r is 0, 1, 2 or 3;
s is 0, 1, 2 or 3; and
j is 0, 1 or 2.

2. The compound of formula (I) or a pharmaceutically acceptable salt thereof according to claim 1, being a compound of formula (II) or a pharmaceutically acceptable salt thereof: 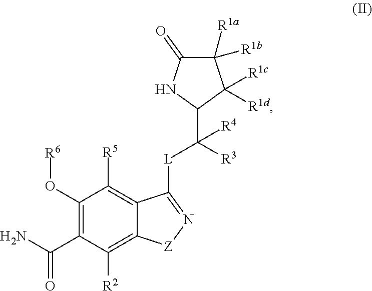
wherein:
R^{1a}, R^{1b}, R^{1c} and R^{1d} are identical or different and are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b} and cycloalkyl;
Z, L, R²-R⁶, r, R^{a} and R^{b} are each as defined in claim 1.

3. The compound of formula (I) or a pharmaceutically acceptable salt thereof according to claim 1, wherein L is O.

4. The compound of formula (I) or a pharmaceutically acceptable salt thereof according to claim 1, being a compound of formula (III) or a pharmaceutically acceptable salt thereof: 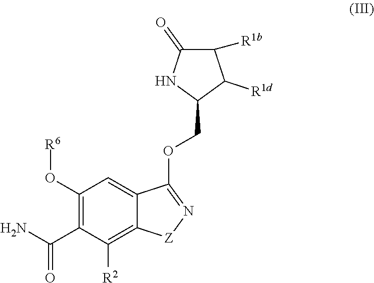
wherein:
R^{1b} and R^{1d} are identical or different and at each occurrence are independently selected from hydrogen, halogen, alkyl, haloalkyl, alkoxy, alkylthio, haloalkoxy, haloalkylthio, hydroxy, hydroxyalkyl, cyano, amino, —(CH₂)ᵣ—NR^{a}R^{b} and cycloalkyl; and
Z, R⁶, R^{a}, R^{b} and r are each as defined in claim 1.

5. The compound of formula (I) or a pharmaceutically acceptable salt thereof according to claim 1, wherein Z is S.

6. The compound of formula (I) or a pharmaceutically acceptable salt thereof according to claim 1, wherein R¹ is selected from hydrogen, halogen, C₁₋₆ alkyl and oxo.

7. The compound of formula (I) or a pharmaceutically acceptable salt thereof according to claim 1, wherein R² is hydrogen, halogen or C₁₋₆ alkyl.

8. The compound of formula (I) or a pharmaceutically acceptable salt thereof according to claim 1, wherein R³ and R⁴ are independently hydrogen or C₁₋₆ alkyl.

9. The compound of formula (I) or a pharmaceutically acceptable salt thereof according to claim 1, wherein R⁵ is hydrogen, halogen or C₁₋₆ alkyl.

10. The compound of formula (I) or a pharmaceutically acceptable salt ereof according to claim 1, wherein each R⁶ is C₁₋₆ alkyl; preferably methyl.

11. The compound of formula (I) or a pharmaceutically acceptable salt thereof according to claim 1, wherein R⁷ and R⁸ are independently hydrogen or C₁₋₆ alkyl.

12. The compound of formula (I) or a pharmaceutically acceptable salt thereof according to claim 1, wherein the compound is selected from:

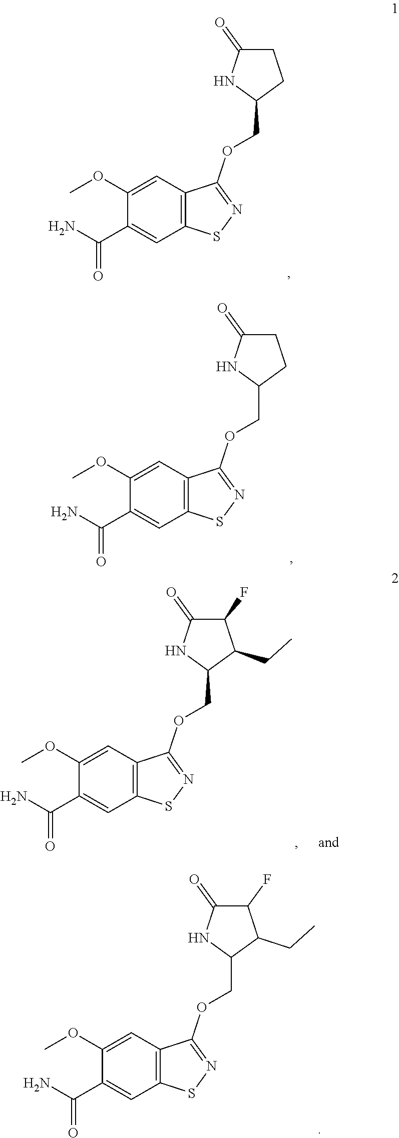 [SMILES: COc1cc2c(OC[C@@H]3CCC(=O)N3)nsc2cc1C(N)=O ; COc1cc2c(OCC3CCC(=O)N3)nsc2cc1C(N)=O ; CC[C@@H]1[C@H](F)C(=O)N[C@@H]1COc1nsc2cc(C(N)=O)c(OC)cc12 ; CCC1C(COc2nsc3cc(C(N)=O)c(OC)cc23)NC(=O)C1F]

13. A compound of formula (IA) or a pharmaceutically acceptable salt thereof: 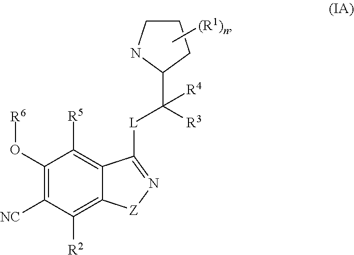
wherein:
Z, L, R¹-R⁶ and n are each as defined in claim 1.

14. The compound of formula (IA) or a pharmaceutically acceptable salt thereof according to claim 13, wherein the compound is selected from:

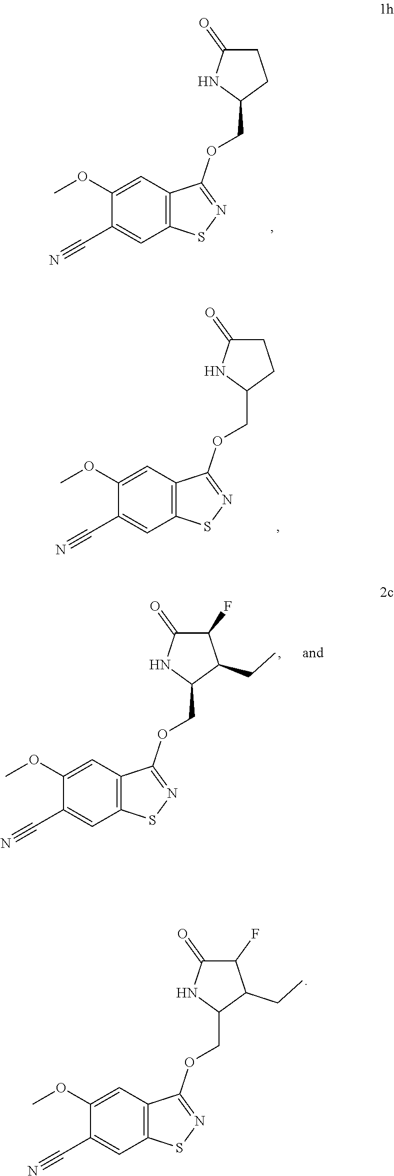

15. A process of preparing the compound of formula (I) according to claim 1, comprising a step of: 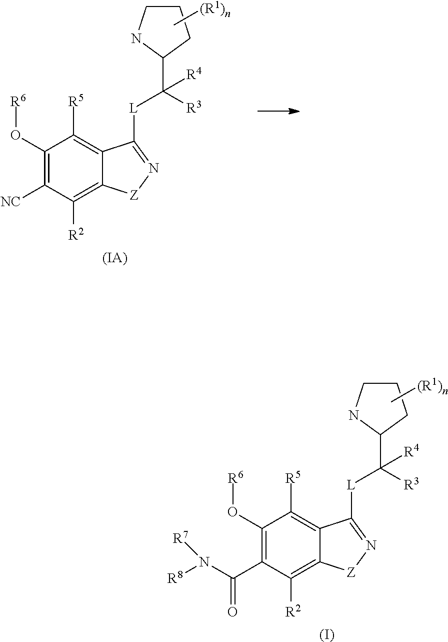
subjecting the compound of formula (IA) to a hydration reaction to obtain the compound of formula (I), wherein:
R⁷ is hydrogen;
R⁸ is hydrogen; and
Z, L, to R⁶ and n are each as defined in claim 1.

16. A pharmaceutical composition, comprising a compound according to claim 1 or a pharmaceutically acceptable salt thereof, and a pharmaceutically acceptable carrier.

17. A method of inhibiting IRAK protein kinase, e.g., an ERAK-4 protein kinase, comprising administering to a subject in need thereof a compound according to claim 1 or a pharmaceutically acceptable salt thereof, or a pharmaceutical composition thereof.

18. A method of preventing and/or treating an IRAK-mediated disorder, disease, or condition, comprising administering to a subject in need thereof a therapeutically effective amount of a compound according to claim 1 or a pharmaceutically acceptable salt thereof, wherein the IRAK-mediated disorder, disease or condition is selected from a cancer, a neurodegenerative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, and a CNS disorder.

19. The method according to claim 18, wherein the cancer or proliferative disorder is selected from a benign or malignant tumor, solid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, nonsmall-cell lung carcinoma, lymphomas, Hodgkins and Non-Hodgkins, a mammary carcinoma, follicular carcinoma, undifferentiated carcinoma, papillary carcinoma, seminoma, melanoma, an IL-1 driven disorder, an MyD88 driven disorder, Smoldering of indolent multiple myeloma, or hematological malignancies (including leukemia, diffuse large B-cell lymphoma (DLBCL), ABCDLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulmemia (WM), splenic marginal zone lymphoma, multiple myeloma, plasmacytoma, and intravascular large B-cell lymphoma).

20. The method according to claim 19, wherein the MyD88 driven disorder is selected from ABC DLBCL, Waldenstrom's macroglobulmemia, Hodgkin's lymphoma, primary cutaneous T-cell lymphoma and chronic lymphocytic leukemia; and/or the IL-1 driven disorder is Smoldering of indolent multiple myeloma; and/or the neurodegenerative disease is selected from Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease; and/or the inflammatory disorder is selected from conditions of the eye such as ocular allergy, conjunctivitis, keratoconjunctivitis sicca, and vernal conjunctivitis; diseases affecting the nose including allergic rhinitis; and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, nephritis, vasculitis, diverticulitis, interstitial cystitis, glomerulonephritis (with and without nephrotic syndrome, e.g. including idiopathic nephrotic syndrome or urinal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, vulvitis, alopecia areata, erythema multiform, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acute and chronic gout, chronic gouty arthritis, psoriasis, psoriatic arthritis, rheumatoid arthritis, Juvenile rheumatoid arthritis, Cryopyrin Associated Periodic Syndrome (CAPS), and osteoarthritis.
